(19) United States
(12) Patent Application Publication
(10) Pub. No.: US 2010/0016352 A1
(43) Pub. Date: Jan. 21, 2010

(54) COMPOUNDS AND METHODS FOR TREATING ESTROGEN RECEPTOR-RELATED DISEASES

(76) Inventors: Jin Li, Pawcatuck, CT (US); Kun Meng, Hong Kong (CN)

Correspondence Address: PERKINS COIE LLP, POST OFFICE BOX 1208, SEATTLE, WA, 98111-1208, US

(21) Appl. No.: 12/558,392

(22) Filed: Sep. 11, 2009

Related U.S. Application Data
(63) Continuation-in-part of application No. PCT/US09/40775, filed on Apr. 16, 2009.
(60) Provisional application No. 61/046,255, filed on Apr. 18, 2008.

(51) Int. Cl. A61K 31/47; A61K 31/353; A61P 35/00

(52) U.S. Cl. 514/312; 514/456

(57) ABSTRACT

Provided herein in certain embodiments are compounds, pharmaceutical compositions and methods for modulating the functions of estrogen receptor alpha 36, for preventing and/or treating diseases mediated by estrogen receptor alpha 36, for preventing and/or treating osteoporosis, for inducing cell death and/or inhibiting cell proliferation and for preventing and/or treating diseases involving abnormal cell proliferation such as cancers.

### CROSS-REFERENCE TO RELATED APPLICATION

This is a continuation in part application of International PCT Application No. PCT/US09/40775, filed on Apr. 16, 2009 and designating the United States, which claims priority to U.S. provisional application No. 61/046,255, filed on Apr. 18, 2008, both of which are hereby incorporated by reference in their entirety.

### FIELD OF THE INVENTION

The present invention relates to compounds, pharmaceutical compositions and methods for preventing and/or treating estrogen receptor-related diseases.

### BACKGROUND

Estrogens are a group of hormones that are involved in many critical physiological functions in the human body. Estrogen functions include developing the female sex organs, preparing the breast and uterus for pregnancy and breast feeding after childbirth. Estrogens also play important roles in maintaining proper cardiovascular function and bone density. Estrogens are known to stimulate cell proliferation and may increase a woman's risk of developing cancers, especially breast cancer and uterus cancer.

Estrogens bind to estrogen receptors in target cells to regulate cell functions. Two types of estrogen receptors were discovered in human cells (hERs), hER-α and hER-β. They share common protein structures, each possessing three independent but interacting functional domains: the N-terminal domain (A/B domain), the central DNA-binding domain (C domain), and the C-terminal ligand-binding domain (D/E/F domain). The N-terminal domain has a ligand-independent activation function (AF-1), which is involved in interaction with co-activators and transcriptional activation of target genes in the absence of ligands. The DNA binding-domain plays important roles in receptor dimerization and binding to specific DNA sequences. The C-terminal ligand binding-domain mediates ligand binding and has a ligand-dependent transactivation function (AF-2), activating gene transcription in the presence of ligands.

The full-length hER-αwas identified as a 66 kDa protein and referred to as hER-α66. hER-α66 contains all three functional domains. A splice variant of hER-α66 was later discovered and named hER-α46. hER-α46 has a molecular weight of about 46 KDa and lacks the N-terminal AF-1 domain of hER-α66. Recently, a novel 36 kDa hER-αvariant, hER-α36, was identified. It lacks the N-terminal AF-1 domain and the C-terminal AF-2 domain of hER-α66 (Wang et al., Biochem. Biophys. Res. Commun. 336, 1023-1027 (2005)).

hER-α66 is believed to mediate estrogen-stimulated cell proliferation via transcriptional activation of its target genes. Binding of estrogen to hER-α66 activates the transactivation domain of hER-α66 and thus stimulates the expression of downstream target genes and eventually leads to cell proliferation. hER-α46 was found to mediate membrane-initiated and estrogen-stimulated rapid NO synthesis (Li et al., Proc. Natl. Acad. Sci. USA 100: 4807-4812 (2003)). It was also shown that hER-α46, that lacks the AF-1 domain, inhibits the AF-1 activity of hER-α66 (Flouriot, G., EMBO, 19, 4688-4700, (2000)). Since hER-α36 lacks both the AF-1 and AF-2 transcriptional activation domains, it functions as a dominant-negative inhibitor of hER-α66 and hER-β to inhibit both AF-1 and AF-2 functions of hER-α and hER-β. In addition, hER-α36 is localized primarily on the plasma membrane and mediates membrane-initiated mitogenic estrogen signaling that stimulates cell proliferation. (Wang et al., Biochem. Biophys. Res. Commun. 336, 1023-1027 (2005); Wang et al., Proc. Natl. Acad. Sci. U.S.A. 103: 9063-9068 (2006)).

Extensive studies have shown that estrogen signaling is mediated via the classic nuclear transcriptional activation pathways as well as the non-classic membrane-initiated signaling pathways. It seems that hER-α66 and hER-α46 function primarily in the nucleus while hER-α36 functions mainly through outside of the nucleus

It was also shown that hER-α36 lacks Helix 8-12 of the ligand-binding domain of the original hER-α66, which totally changes the ligand binding specificity of hER-α36. Thus, hER-α36 may bind to different ligands from hER-α66 and hER-β.

As estrogen and estrogen receptor related diseases continue to affect many individuals, there remains an urgent need to discover novel compounds and methods useful to prevent and/or treat such diseases.

### SUMMARY

One embodiment of the invention provides compounds, derivatives thereof, pharmaceutical compositions and methods for modulating the functions of the novel estrogen receptor variant, ER-α36. Another embodiment of the invention provides compounds, derivatives thereof, pharmaceutical compositions and methods for preventing and/or treating diseases mediated by ER-α36. Another embodiment of the invention provides compounds, derivatives thereof, pharmaceutical compositions and methods for inducing cell death and/or inhibiting cell proliferation, and for preventing and/or treating diseases involving abnormal cell proliferation such as cancer. Further, another embodiment of the present invention provides compounds, derivatives thereof, pharmaceutical compositions and methods for preventing and/or treating osteoporosis, asthma and other respiratory diseases.

Certain embodiments of the invention provide compounds for modulating the function of ER-α36. Certain embodiments of the invention provide methods of modulating the function of ER-α36 using the compounds of the invention. Certain embodiments of the invention provide methods of preventing and/or treating a disease mediated by the functions or dysfunctions of ER-α36.

Certain embodiments of the invention provide compounds for inducing cell death. Certain embodiments of the invention provide methods of inducing cell death using the compounds of the invention.

Certain embodiments of the invention provide compounds for inhibiting cell proliferation. Certain embodiments of the invention provide methods of inhibiting cell proliferation using the compounds of the invention.

Certain embodiments of the invention provide compounds for preventing and/or treating a disease involving abnormal cell proliferation. Certain embodiments of the invention provide methods of preventing and/or treating a disease involving abnormal cell proliferation in a subject using the compounds of the invention.

Certain embodiments of the invention provide compounds for preventing and/or treating asthma and other respiratory diseases. Certain embodiments of the invention provide methods of preventing and/or treating asthma and other respiratory diseases in a subject using the compounds of the invention.

Certain embodiments of the invention provide compounds for preventing and/or treating osteoporosis. Certain embodiments of the invention provide methods of preventing and/or treating osteoporosis in a subject using the compounds of the invention.

Certain embodiments of the present invention provide pharmaceutical compositions comprising the compounds of the invention.

### BRIEF DESCRIPTION OF THE FIGURES

FIG. 1 shows Western blot results depicting the expression of ER-α66, ER-α46 and ER-α36 in human breast cancer samples. Lane 1: normal breast tissue; Lane 2: infiltrating ductal carcinoma; Lane 3: infiltrating ductal carcinoma; Lane 4: invasive ductal carcinoma; Lane 5: infiltrating lobular carcinoma; Lane 6: infiltrating lobular carcinoma; Lane 7: non-invasive ductal carcinoma.

FIG. 2 shows the immunofluorescence staining result of MDA-MB-231 cells, an ER-negative breast cancer cell line that lacks ER-α66 and ER-α46, stained with an antibody that specifically binds to ER-α36 (shown in the figure labeled with “ER-α36”: positive staining shown in green). Cell nucleus was also stained with 4,6-Diamidine-2-Phenylindole (shown in the lane labeled with “DAPI”: positive staining shown in blue). Merged staining signals were shown in lane labeled with “Merge”. Negative staining was observed when the antibody was pre-incubated with immunogen peptides that bind to the antibody.

### DETAILED DESCRIPTION

The Compounds and Derivatives Thereof

Provided herein in certain embodiments are compounds, derivatives thereof, and pharmaceutical compositions useful for modulating the functions of the novel estrogen receptor, ER-α36, preventing and/or treating diseases mediated by ER-α36, inducing cell death, inhibiting cell proliferation, preventing and/or treating diseases involving abnormal cell proliferation such as cancer, and/or preventing and/or treating osteoporosis, asthma and other respiratory diseases.

In certain embodiments, compounds of Formula (I):

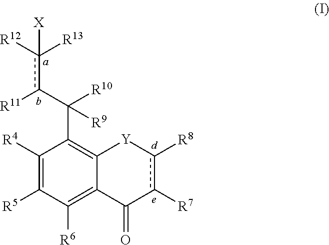

a stereoisomer or prodrug thereof, or a pharmaceutically acceptable salt of said compound, stereoisomer, or prodrug, are provided wherein:
X═H, OR¹ or NR²R³; Y═NR, O; R¹, R², R and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R⁸ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, aryl (including substituted aryl), (C₆-C₁₀)aryloxy, heteroaryl (including substituted heteroaryl), (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹², and R¹³ are hydrogen, halo, hydroxyl, (C₁-C₆)alkyl,
When R, R¹, R², R³, R⁴, R⁵, R⁶, R⁷, R⁸, R⁹, R¹⁰, R¹¹, R¹², R¹³ are (C₁-C₆)alkyl group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N—(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₈)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

One embodiment of the present invention includes a group of compounds of Formula (I) referred to as the IA1 group of compounds, wherein said group of compounds have the formula:

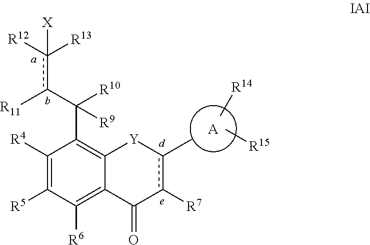

Wherein:

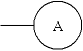

is (C₆-C₁₀)aryl, (C₅-C₈)heteroaryl, X═H, OR¹ or NR²R³; Y═NR, O; wherein R, R¹, R² and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R¹⁴ and R¹⁵ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl (including substituted aryl), (C₆-C₁₀)aryloxy, (C₅-C₉)heteroaryl (including substituted heteroaryl), (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹² and R¹³ are hydrogen, halo, hydroxyl, (C₁-C₆)alkyl;
   When R, R¹, R², R³, R⁴, R⁵, R⁶, R⁷, R⁹, R¹⁰, R¹¹, R¹², R¹³, R¹⁴ and R¹⁵ are (C₁-C₆)alkyl group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N—(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₉)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

One embodiment of the present invention includes a group of compounds of Formula (I) referred to as the IA2 group of compounds, wherein said group of compounds have the formula:

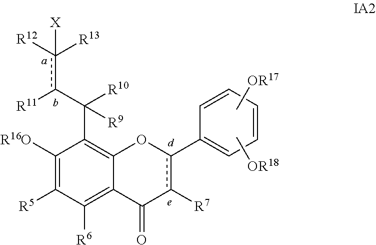

Wherein R¹⁶, R¹⁷ and R¹⁸ and are independently hydrogen, (C₁-C₆)alkyl, (C₂-C₆)alkenyl, (C₂-C₆)alkynyl; the bond between carbon a and b or d and e may be single or double bond. R⁵, R⁶, R⁷, R⁹, R¹⁰, R¹¹, R¹², R¹³ and X are as defined above.

Another embodiment of the present invention includes a group of compounds of Formula (I) referred to as the IA3 group of compounds, wherein said group of compounds have the formula:

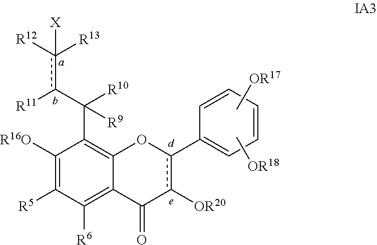

Wherein R¹⁶, R¹⁷, R¹⁸ and R²⁰ are independently hydrogen, (C₁-C₆)alkyl; R⁹, R¹⁰, R¹¹, R¹², R¹³ are independently hydrogen, (C₁-C₆)alkyl; and X are H, OR¹ or NR²R³, R¹, R² and R³ are independently hydrogen, (C₁-C₆)alkyl or R²R³ together as —(CH₂)ₙ—, n=2 to 8; the bond between carbon a and b or d and e may be single or double bond. R⁵ and R⁶ are defined as above.

Another embodiment of the present invention includes a group of compounds of Formula (I) referred to as the IA4 group of compounds, wherein said group of compounds have the formula:

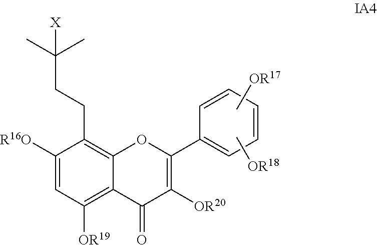

Wherein R¹⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R²R³ together as —(CH₂)ₙ—, n=2 to 5;

Another embodiment of the present invention includes a group of compounds of Formula (I) referred to as the IA5 group of compounds, wherein said group of compounds have the formula:

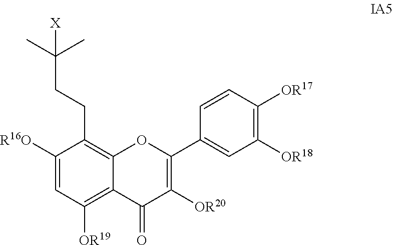

Wherein R⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R₂R₃ together as —(CH₂)ₙ—, n=2 to 5;

Another embodiment of the present invention includes a group of compounds of Formula (I) referred to as the IA6 group of compounds, wherein said group of compounds have the formula:

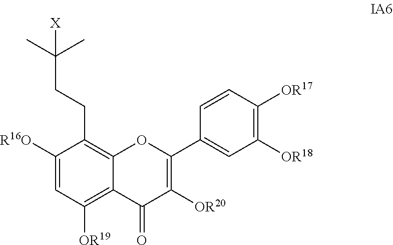

Wherein R¹⁶, R¹⁷, R¹⁸, R¹⁹ and R²⁰ are independently hydrogen, (C₁-C₆)alkyl; and X are OH or NH₂.

Examples of specific preferred compounds of Formula (I) include but are not limited to the following:
8-(3-amino-3-methylbutyl)-3,5,7-trihydroxy-2-(4-hydroxyphenyl)-4H-chromen-4-one
8-(3-amino-3-methylbutyl)-3,5,7-trihydroxy-2-(4-methoxyphenyl)-4H-chromen-4-one
8-(3-amino-3-methylbutyl)-5,7-dihydroxy-3-methoxy-2-(4-methoxyphenyl)-4H-chromen-4-one
5,7-dihydroxy-8-(3-hydroxy-3-methylbutyl)-3-methoxy-2-(4-methoxyphenyl)-4H-chromen-4-one
5,7-dihydroxy-8-(3-hydroxy-3-methylbutyl)-3-methoxy-2-(3,4-dimethoxyphenyl)-4H-chromen-4-one
5-hydroxy-8-(3-hydroxy-3-methylbutyl)-3,7-dimethoxy-2-(3,4-dimethoxyphenyl)-4H-chromen-4-one
8-(3-amino-3-methylbutyl)-5-hydroxy-3,7-dimethoxy-2-(3,4-dimethoxyphenyl)-4H-chromen-4-one
3-(2-(piperidin-1-yl)ethoxy)-5,7-dihydroxy-8-(3-hydroxy-3-methylbutyl)-2-(4-methoxyphenyl)-4H-chromen-4-one
3-(2-(piperidin-1-yl)ethoxy)-8-(3-amino-3-methylbutyl)-5,7-dihydroxy-2-(4-methoxyphenyl)-4H-chromen-4-one
2-(4-chlorophenyl)-3,5,7-trihydroxy-8-(3-hydroxy-3-methylbutyl)-4H-chromen-4-one
2-(4-chloro-3-methoxyphenyl)-3,5,7-trihydroxy-8-(3-hydroxy-3-methylbutyl)-4H-chromen-4-one
8-(3-amino-3-methylbutyl)-3,5,7-trihydroxy-2-(pyridin-3-yl)-4H-chromen-4-one
8-(3-amino-3-methylbutyl)-3,5,7-trihydroxy-2-(pyridin-2-yl)-4H-chromen-4-one
8-(3-amino-3-methylbutyl)-5,7-dihydroxy-3-methoxy-2-(5-methoxypyridin-2-yl)-4H-chromen-4-one
5,7-dihydroxy-8-(3-hydroxy-3-methylbutyl)-3-methoxy-2-(6-methoxypyridin-3-yl)-4H-chromen-4-one
5,7-dihydroxy-3-methoxy-8-(3-methylbut-2-enyl)-2-(pyridin-3-yl)-4H-chromen-4-one
2-(6-(dimethylamino)pyridin-3-yl)-5,7-dihydroxy-3-methoxy-8-(3-methylbut-2-enyl)-4H-chromen-4-one
2,3-dihydro-5,7-dihydroxy-2-(4-methoxyphenyl)-8-(3-methylbut-2-enyl)quinolin-4(1H)-one
2,3-dihydro-7-hydroxy-2-(4-methoxyphenyl)-8-(3-methylbut-2-enyl)quinolin-4(1H)-one
7-hydroxy-2-(4-methoxyphenyl)-8-(3-methylbut-2-enyl)quinolin-4(1H)-one
5,7-dihydroxy-2-(4-methoxyphenyl)-8-(3-methylbut-2-enyl)quinolin-4(1H)-one
8-(3-amino-3-methylbutyl)-5,7-dihydroxy-2-(4-hydroxyphenyl)-4H-chromen-4-one
8-(3-amino-3-methylbutyl)-5,7-dihydroxy-2-(4-methoxyphenyl)-4H-chromen-4-one
8-(3-amino-3-methylbutyl)-2,3-dihydro-5,7-dihydroxy-2-(4-methoxyphenyl)chromen-4-one
5,7-dihydroxy-8-(3-hydroxy-3-methylbutyl)-2-(4-methoxyphenyl)-4H-chromen-4-one
5,7-dihydroxy-8-(3-hydroxy-3-methylbutyl)-2-(3,4-dimethoxyphenyl)-4H-chromen-4-one
5-hydroxy-8-(3-hydroxy-3-methylbutyl)-7-methoxy-2-(3,4-dimethoxyphenyl)-4H-chromen-4-one
8-(3-amino-3-methylbutyl)-5-hydroxy-7-methoxy-2-(3,4-dimethoxyphenyl)-4H-chromen-4-one
2,3-dihydro-5,7-dihydroxy-8-(3-hydroxy-3-methylbutyl)-2-(4-methoxyphenyl)chromen-4-one
2-(4-aminophenyl)-5,7-dihydroxy-8-(3-hydroxy-3-methylbutyl)-4H-chromen-4-one
2-(4-chlorophenyl)-5,7-dihydroxy-8-(3-hydroxy-3-methylbutyl)-4H-chromen-4-one
2-(4-chloro-3-methoxyphenyl)-5,7-dihydroxy-8-(3-hydroxy-3-methylbutyl)-4H-chromen-4-one
2-(4-aminophenyl)-2,3-dihydro-5,7-dihydroxy-8-(3-hydroxy-3-methylbutyl)chromen-4-one
2-(4-chlorophenyl)-2,3-dihydro-5,7-dihydroxy-8-(3-hydroxy-3-methylbutyl)chromen-4-one
2-(4-chloro-3-methoxyphenyl)-2,3-dihydro-5,7-dihydroxy-8-(3-hydroxy-3-methylbutyl)chromen-4-one
7-hydroxy-8-(3-methylbut-2-enyl)-2-(pyridin-4-yl)-4H-chromen-4-one
7-hydroxy-8-(3-methylbut-2-enyl)-2-(pyridin-3-yl)-4H-chromen-4-one

The compounds and derivatives provided herein may be named according to either the IUPAC (International Union for Pure and Applied Chemistry) or CAS (Chemical Abstracts Service, Columbus, Ohio) nomenclature systems.

The carbon atom content of the various hydrocarbon-containing moieties herein may be indicated by a prefix designating the minimum and maximum number of carbon atoms in the moiety, for example, the prefix (Cₐ-C_{b})alkyl indicates an alkyl moiety of the integer “a” to “b” carbon atoms, inclusive. Thus, for example, (C₁-C₆)alkyl refers to an alkyl group of one to six carbon atoms inclusive.

The term “alkoxy” refers to straight or branched, monovalent, saturated aliphatic chains of carbon atoms bonded to an oxygen atom. Examples of alkoxy groups include methoxy, ethoxy, propoxy, butoxy, iso-butoxy, tert-butoxy, and the like.

The term “alkyl” refers to straight or branched, monovalent, saturated aliphatic chains of carbon atoms and includes, for example, methyl, ethyl, propyl, isopropyl, butyl, isobutyl, pentyl, isopentyl, hexyl, and the like.

The term “alkenyl” denotes a straight or branched-chain hydrocarbon having one or more double bonds and includes, for example, vinyl, allyl, and the like.

The term “aryl” denotes a cyclic, aromatic hydrocarbon. Examples of aryl groups include phenyl, naphthyl, anthracenyl, phenanthrenyl, and the like.

The term “cycloalkyl” denotes a saturated monocyclic or polycyclic cycloalkyl group, optionally fused to an aromatic hydrocarbon group. Examples of cycloalkyl groups include cyclopropyl, cyclobutyl, cyclopentyl, cyclohexyl, cycloheptyl, indanyl, tetrahydronaphthalinyl, and the like.

The term “halogen” or “halo” represents chloro, bromo, fluoro, and iodo atoms.

The term “heteroaryl” denotes a monocyclic or polycyclic aromatic hydrocarbon group wherein one or more carbon atoms have been replaced with heteroatoms such as nitrogen, oxygen, or sulfur. If the heteroaryl group contains more than one heteroatom, the heteroatoms may be the same or different. Examples of heteroaryl groups include benzofuranyl, benzothienyl, benzimidazolyl, benzoxazolyl, benzothiazolyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyrido[3,4-b]indolyl, pyridyl, pyrimidyl, pyrrolyl, quinolizinyl, quinolyl, quinoxalinyl, thiadiazolyl, thiatriazolyl, thiazolyl, thienyl, triazinyl, triazolyl, xanthenyl, and the like.

The term “heterocycloalkyl” denotes a saturated monocyclic or polycyclic cycloalkyl group, optionally fused to an aromatic hydrocarbon group, in which at least one of the carbon atoms have been replaced with a heteroatom such as nitrogen, oxygen, or sulfur. If the heterocycloalkyl group contains more than one heteroatom, the heteroatoms may be the same or different. Examples of such heterocycloalkyl groups include azabicycloheptanyl, azetidinyl, indolinyl, morpholinyl, piperazinyl, piperidyl, pyrrolidinyl, tetrahydrofuryl, tetrahydroquinolinyl, tetrahydroindazolyl, tetrahydroindolyl, tetrahydroisoquinolinyl, tetrahydropyranyl, tetrahydroquinoxalinyl, tetrahydrothiopyranyl, thiazolidinyl, thiomorpholinyl, thioxanthenyl, thioxanyl, and the like.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “oxo”, means a carbonyl group formed by the combination of a carbon atom(s) and an oxygen atom(s).

The term “prodrug” refers to a compound that is a drug precursor which, following administration to a subject, releases the drug in vivo via a chemical or physiological process (e.g., upon being brought to physiological pH or through enzyme activity). A discussion of the synthesis and use of prodrugs is provided by T. Higuchi and W. Stella, in “Prodrugs as Novel Delivery Systems,” vol. 14 of the ACS Symposium Series, and in Bioreversible Carriers in Drug Design, ed. Edward B. Roche, American Pharmaceutical Association and Pergamon Press, 1987, both of which are incorporated herein by reference. The term “prodrug” may include a metabolic precursor of a compound of the invention. The prodrug may be inactive when administered to a subject but is converted in vivo to a compound of the invention. The prodrug can be naturally existing compounds or synthetic compounds.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “salts” and “pharmaceutically acceptable salts” refers to organic and inorganic salts of a compound of Formula (I), or a stereoisomer, or prodrug thereof. These salts can be prepared in situ during the final isolation and purification of a compound, or by separately reacting a compound of Formula (I), or a stereoisomer, or prodrug thereof, with a suitable organic or inorganic acid or base and isolating the salt thus formed. Representative salts include the hydrobromide, hydrochloride, sulfate, bisulfate, nitrate, acetate, oxalate, besylate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may also include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. For additional examples see, for example, Berge, et al., J. Pharm. Sci., 66, 1-19 (1977), which is incorporated herein by reference.

A salt of a compound of Formula (I) may be readily prepared by mixing together solutions of a compound of Formula (I) and the desired acid or base, as appropriate. The salt may precipitate from solution and be collected by filtration or may be recovered by evaporation of the solvent.

The term “substituted” means that a hydrogen atom on a molecule has been replaced with a different atom or molecule. The atom or molecule replacing the hydrogen atom is denoted as a “substituent.”

The compounds of Formula (I) may contain asymmetric or chiral centers and, therefore, exist in different stereoisomeric forms. It is intended that all stereoisomeric forms of the compounds of Formula (I) as well as mixtures thereof, including racemic mixtures, form part of the present invention. In addition, all geometric and positional isomers are also contemplated. For example, if a compound of Formula (I) incorporates a double bond, both the cis- and frans-forms, as well as mixtures thereof, are embraced within the scope of the invention.

Diasteriomeric mixtures can be separated into their individual diastereomers on the basis of their physical chemical differences by methods well-known to those of ordinary skill in the art, such as by chromatography and/or fractional crystallization. Enantiomers can be separated by converting the enantiomeric mixture into a diasteriomeric mixture by reaction with an appropriate optically active compound (e.g., alcohol), separating the diasteriomers and converting (e.g., hydrolyzing) the individual diasteriomers to the corresponding pure enantiomers. Also, some of the compounds of Formula (I) may be atropisomers (e.g., substituted biaryls) and are also considered as part of the invention.

The compounds of Formula (I) may exist in unsolvated as well as solvated forms with pharmaceutically acceptable solvents, such as water, ethanol, and the like, and it is intended that the invention embrace both solvated and unsolvated forms.

It is also possible that the compounds of Formula (I) may exist as tautomeric isomers in equilibrium, and all such forms are embraced within the scope of the invention.

In certain embodiments, isotopically-labeled compounds of Formula (I), which are identical to those recited herein, but for the fact that one or more atoms are replaced by an atom having an atomic mass or mass number different from the atomic mass or mass number usually found in nature are provided. Examples of isotopes that can be incorporated into compounds of Formula (I) include isotopes of hydrogen, carbon, nitrogen, oxygen, phosphorus, fluorine, and chlorine, such as ²H, ³H, ¹³C, ¹⁴C, ¹⁵N, ¹⁷O, ¹⁸O, ³¹P, ³²P, ³⁵S, ¹⁸F, and ³⁶Cl, respectively. The compounds of Formula (I), the stereoisomers and prodrugs thereof, and the pharmaceutically acceptable salts of the compounds, stereoisomers, or prodrugs, that contain the aforementioned isotopes and/or other isotopes of the other atoms are intended to be within the scope of the instant invention.

Certain isotopically-labeled compounds of Formula (I), for example those compounds into which radioactive isotopes such as ³H and ¹⁴C are incorporated, are useful in compound and/or substrate tissue distribution assays. Tritiated, i.e., ³H, and carbon-14, i.e., ¹⁴C, isotopes are particularly preferred for their relative ease of preparation and facile detection. Furthermore, substitution with heavier isotopes such as deuterium, i.e., ²H, may afford certain therapeutic advantages resulting from greater metabolic stability, for example, increased in vivo half-life, or reduced dosage requirements and, hence, may be preferred in some circumstances. The isotopically-labeled compounds of Formula (I) can generally be prepared by methods known to one of ordinary skill in the art, such as by substituting an isotopically-labeled reagent for a non-isotopically-labeled reagent.

Methods of Use

In certain embodiments, the compounds of the invention are modulators of ER-α36 and are useful for modulating the functions of ER-α36 in cells in vitro and in vivo. The compounds are also useful for preventing and/or treating diseases associated with the functions or dysfunctions of ER-α36. In certain embodiments, the compounds of the invention can induce cell death and/or inhibit cell proliferation and therefore are useful for preventing and/or treating diseases involving abnormal cell proliferation. In certain embodiments, the compounds of the invention are useful for preventing and/or treating osteoporosis, asthma and other respiratory diseases.

In certain embodiments, methods of modulating the functions of ER-α36 in a cell comprising exposing a cell expressing ER-α36 to the compounds of Formula (I) are provided. The cells may express ER-α36 endogenously or exogenously through genetic engineering. In one embodiment, the cells express ER-α36 endogenously. In a preferred embodiment, the cells are cancer cells that express ER-α36 endogenously. Examples of cancer cells that express ER-α36 are breast cancer cells, leukemia cells, lung cancer cells, myeloma cells, prostate cancer cells, ovarian cancer cells, colon cancer cells and stomach cancer cells. In a further preferred embodiment, the cells expressing ER-α36 are breast cancer cells that express ER-α36 endogenously. Examples of breast cancer cells expressing ER-α36 are MCF7 and MDA-MB-231 cells. The expression of the endogenous ER-α36, may be increased or decreased through treatment with one or more agents. Examples of such agents are serum, E2β (17β-estradiol), Tamoxifen and ICI 182,780.

In another embodiment, the cells are altered by genetic engineering to express exogenous ER-α36. Cells expressing exogenous ER-α36 may be prepared by genetic engineering methods known to one of ordinary skill in the art (See Sambrook et al., Molecular Cloning, A Laboratory Manual (2d Ed. 1989) (Cold Spring Harbor Laboratory)). Briefly, an exogenous ER-α36 gene is prepared and inserted into an expression vector, which is transfected into a host cell, which is then grown in a culture solution suitable for expressing the exogenous ER-α36. An example of the gene sequence of human ER-α36 is disclosed in Wang et al., Biochem. Biophys. Res. Commun. 336, 1023-1027 (2005) (GenBank Accession No. BX640939). The cells expressing exogenous ER-α36 may or may not express endogenous ER-α36. The expression levels of endogenous or exogenous ER-α36 in the cells may be increased or decreased by treatment with one or more other agents. Examples of such agents are serum, E2β (17β-estradiol), Tamoxifen and ICI 182,780.

The cells expressing ER-α36 may or may not express other estrogen receptors such as ER-α66, ER-α46 and ER-β.

In certain embodiments, methods of preventing and/or treating a disease mediated by ER-α36 in a subject comprising administering to the subject a pharmaceutical composition comprising the compounds of Formula (I) are provided. Examples of diseases mediated by ER-α36 include without limitation alzheimer's disease; neuron degeneration; neuron aging and damaging, birth control; abortion; bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, perimenopausal depression, post-partum depression, premenstrual syndrome, manic depression, anxiety, dementia, obsessive compulsive behavior, attention deficit disorder, sleep disorders, irritability, impulsivity, immune deficiency, auto immune diseases, anger management, multiple sclerosis and Parkinson's disease, inflammation, inflammatory bowel disease, respiratory diseases, sexual dysfunction, hypertension, retinal degeneration, asthma and cancers. Preferably, diseases mediated by ER-α36 include bone loss, bone fracture, osteoporosis, menopause, premenstrual syndrome, endometriosis, uterine disease, impotence, sexual dysfunctions, increased levels of LDL cholesterol, cardiovascular diseases, vascular smooth muscle cell proliferation, depression resulting from an estrogen deficiency, perimenopausal depression, post-partum depression, immune deficiency, auto immune diseases, inflammation, asthma and cancers. More preferably, diseases mediated by ER-α36 include bone loss, osteoporosis, impotence, cardiovascular diseases, immune deficiency, inflammation, asthma and cancers. The subject may be a mammal such as a dog, cat, cow, sheep, horse, or human, preferably a human. The required therapeutic amount for the method will vary according to the specific diseases and is readily ascertainable by one of ordinary skill in the art having benefit of the instant disclosure.

In certain embodiments, methods of inducing cell death comprising exposing a cell to an effective amount of the compounds of Formula (I) are provided. Furthermore, certain embodiments of the invention provide methods of inhibiting cell proliferation comprising exposing a cell to an effective amount of the compounds of Formula (I). The cells may have normal or abnormal growth. The abnormal cell growth may be benign or malignant. In one embodiment, the cells are cancer cells. In a preferred embodiment the cancer is anal cancer, bile duct cancer, bladder cancer, bone cancer, bowel cancer (colon cancer, rectal cancer), brain cancer, breast cancer, carcinoid cancer, cervix cancer, endocrine cancer, endometrial cancer, eye cancer, gall bladder cancer, head and neck cancer, Kaposi's sarcoma cancer, kidney cancer, larynx cancer, leukemia, liver cancer, lung cancer, lymphoma, melanoma, mesothelioma, myeloma, neuroendocrine cancer, oesophagus cancer, ovary cancer, pancreas cancer, penis cancer, prostate cancer, skin cancer, soft tissue sarcomas cancer, spinal cord cancer, stomach cancer, testes cancer, thyroid cancer, vagina cancer, vulva cancer, or uterus cancer. In a further preferred embodiment, the cancer is breast cancer, cervix cancer, colon cancer, endometrial cancer, leukemia, liver cancer, lung cancer, myeloma, ovary cancer, prostate cancer, stomach cancer, or uterus cancer. In an even further preferred embodiment, the cancer is breast cancer, cervix cancer, endometrial cancer, lung cancer, uterus cancer or prostate cancer. In certain embodiments, the cells may express estrogen receptors, in particular, ER-α36, endogenously or exogenously. In a preferred embodiment, the cells express ER-α36 endogenously.

The effective amount of the compounds of Formula (I) for inducing cell death and/or inhibiting cell proliferation will vary according to the specific cell types and treatment conditions. It is readily ascertainable by one of ordinary skill in the art having benefit of the instant disclosure. In one embodiment, the effective amount of the compounds of Formula (I) that the cell is exposed to is a concentration of at least about 0.1 μM. In another embodiment, the concentration of the compounds of Formula (I) that the cell is exposed to is within the range of about 0.1 μM to 100 μM. Preferably, the effective amount is a concentration of the compounds within the range of about 5 μM to 50 μM or about 5 μM to 30 μM or about 5 μM to 25 μM or about 5 μM to 20 μM or about 5 μM to 10 μM.

In certain embodiments, methods of preventing and/or treating a disease involving abnormal cell proliferation in a subject comprising administering to the subject a therapeutically effective amount of a pharmaceutical composition comprising the compounds of Formula (I) are provided.

The abnormal cell proliferation may be benign cell growth or cancerous. In one embodiment, the disease involving abnormal cell proliferation is cancer. In a preferred embodiment the cancer is anal cancer, bile duct cancer, bladder cancer, bone cancer, bowel cancer (colon cancer, rectal cancer), brain cancer, breast cancer, carcinoid cancer, cervix cancer, endocrine cancer, endometrial cancer, eye cancer, gall bladder cancer, head and neck cancer, Kaposi's sarcoma cancer, kidney cancer, larynx cancer, leukemia cancer, liver cancer, lung cancer, lymphoma cancer, melanoma cancer, mesothelioma cancer, myeloma cancer, neuroendocrine cancer, oesophagus cancer, ovary cancer, pancreas cancer, penis cancer, prostate cancer, skin cancer, soft tissue sarcomas cancer, spinal cord cancer, stomach cancer, testes cancer, thyroid cancer, vagina cancer, vulva cancer, or uterus cancer. In a further preferred embodiment, the cancer is breast cancer, cervix cancer, colon cancer, endometrial cancer, leukemia, liver cancer, lung cancer, myeloma, ovary cancer, prostate cancer, stomach cancer, or uterus cancer. In an even further preferred embodiment, the cancer is breast cancer, cervix cancer, endometrial cancer, lung cancer, uterus cancer or prostate cancer.

In certain embodiments, methods of preventing and/or treating asthma and other respiratory diseases in a subject comprising administering to the subject a therapeutically effective amount of a pharmaceutical composition comprising the compounds of Formula (I) are provided. Asthma refers to inflammatory disorders of the airways with reversible airflow obstruction. Other respiratory diseases may include disorders of the respiratory tracts and lung such as bronchitis, cystic fibrosis, emphysema, pneumonia, rhinitis and sinusitis.

The subject is preferably a mammal. In one embodiment, the mammal is a dog, cat, cow, sheep, horse, or human. In a preferred embodiment, the mammal is a human.

The compounds of Formula (I) may be administered to a subject by any method that enables delivery of the compounds to the site of action. These methods include, without limitation, oral, buccal, sublingual, ocular, topical (e.g., transdermal), parenteral (e.g., intravenous, intramuscular, or subcutaneous, intravascular or infusion), rectal, intracisternal, intravaginal, intraperitoneal, intravesical, or nasal methods.

The compounds of Formula (I) may be administered to a subject at dosage levels in the range of from about 0.1 mg to about 3,000 mg per day, preferably from about 0.1 mg to about 1,000 mg per day, or from about 1 mg to about 500 mg per day, or from about 1 mg to about 300 mg per day, or from about 10 mg to about 300 mg per day, or from about 10 mg to about 200 mg per day, or from about 20 mg to about 200 mg per day, or from about 30 mg to about 200 mg per day, or from about 40 mg to about 200 mg per day, or from about 50 mg to about 200 mg per day, or from about 50 mg to about 100 mg per day. For a normal adult human having a body mass of about 70 kg, a dosage in the range of from about 0.01 mg to about 100 mg per kg body mass is typically sufficient, and preferably from about 0.1 mg to about 100 mg per kg, or from about 0.5 mg to about 100 mg per kg, or from about 1 mg per kg to about 100 mg per kg, or from about 1 mg per kg to about 75 mg per kg, or from about 1 mg per kg to about 50 mg per kg, or from about 1 mg per kg to about 25 mg per kg, or from about 1 mg per kg to about 10 mg per kg, or from about 2 mg per kg to about 5 mg per kg. However, some variability in the general dosage range may be required depending upon the age and mass of the subject being treated, the intended route of administration, the particular compound being administered, and the like. The determination of dosage ranges and optimal dosages for a particular mammalian subject is within the ability of one of ordinary skill in the art having benefit of the instant disclosure.

In certain embodiments, one or more compounds of the invention may be used in combination with one another. Optionally, the compounds of the invention may also be used in combination with any other active agents for modulating cell functions or treating diseases. If a combination of active compounds is used, they may be administered simultaneously, separately or sequentially.

In certain embodiments, the compounds of the invention may be used in combination with one or more other anticancer agents. Suitable anticancer agents include, but are not limited to, alkylating agents, nitrogen mustards, folate antagonists, purine antagonists, pyrimidine antagonists, spindle poisons, topoisomerase inhibitors, apoptosis inducing agents, angiogenesis inhibitors, podophyllotoxins, nitrosoureas, antimetabolites, protein synthesis inhibitors, kinase inhibitors, antiestrogens, cisplatin, carboplatin, interferon, asparginase, leuprolide, flutamide, megestrol, mitomycin, bleomycin, doxorubicin, irinotecan and taxol. In one embodiment, the anticancer agents are antiestrogens such as tamoxifen and ICI 182,780.

The compounds of the invention can be tested for their ability to induce cell death or inhibit cell proliferation using recombinant cells expressing exogenous ER-α36. To make the recombinant cells, an exogenous ER-α36 gene is prepared and inserted into an expression vector, then host cells that do not express or express low level of endogenous ER-α36 are transfected with the expressing vector and stably transfected host cells are selected as the recombinant cells for the testing assay. The recombinant cells are incubated with or without the compounds of the invention. The numbers of cells surviving in the assays with or without the treatment of the compounds of the invention are compared. When the number of cells surviving in the assays with the treatment of the test compound are lower (with statistical significance) than the number of cells surviving in the assays without the test compound, the test compound can induce cell death and/or inhibit cell proliferation.

The recombinant cells discussed above can also be used to test compounds of the invention for their ability to modulate ER-α36 functions. The recombinant cells expressing exogenous ER-α36 and the non-transfected host cells are treated with the test compound under the same conditions. The functions of ER-α36 of interest are observed and analyzed with methods known to one with ordinary skill in the art. Such functions include but are not limited to ER-α36's ability to stimulate its downstream signal transduction pathways such as activation of the Mitogen-Activated Protein kinase (the MAPK/ERK) pathway or the Jun NH2-terminal Kinases (JNKs) pathway.

Pharmaceutical Compositions

In certain embodiments of the methods of the present invention, a compound of Formula (I), a stereoisomer or prodrug thereof, or a pharmaceutically acceptable salt of the compound, stereoisomer, or prodrug, may be administered in the form of a pharmaceutical composition comprising a pharmaceutically acceptable carrier, vehicle, or diluent. Accordingly, a compound of Formula (I), a stereoisomer or prodrug thereof, or a pharmaceutically acceptable salt of the compound, stereoisomer, or prodrug, may be administered to a subject separately or together in any conventional dosage form, including, oral, buccal, sublingual, ocular, topical, parenteral, rectal, intracisternal, intravaginal, intraperitoneal, intravesical, local (e.g., powder, ointment, or drop), or nasal dosage forms.

Pharmaceutical compositions suitable for parenteral injection may comprise pharmaceutically acceptable sterile aqueous or nonaqueous solutions, dispersions, suspensions, or emulsions, and sterile powders for extemporaneous reconstitution into sterile injectable solutions or dispersions. Examples of suitable aqueous and nonaqueous carriers, vehicles, and diluents include water, ethanol, polyols (such as propylene glycol, polyethylene glycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

In certain embodiments, pharmaceutical compositions of the invention may further comprise adjuvants, such as preserving, wetting, emulsifying, and dispersing agents. Prevention of microorganism contamination of the instant compositions can be accomplished with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions may be affected by the use of agents capable of delaying absorption, for example, aluminum monostearate and gelatin.

Solid dosage forms for oral administration can include capsules, tablets, powders, and granules. In certain embodiments of such solid dosage forms, the active compound is admixed with at least one inert conventional pharmaceutical excipient (or carrier) such as sodium citrate or dicalcium phosphate, or (a) fillers or extenders, such as for example, starches, lactose, sucrose, mannitol, or silicic acid; (b) binders, such as for example, carboxymethyl-cellulose, alginates, gelatin, polyvinylpyrrolidone, sucrose, or acacia; (c) humectants, such as for example, glycerol; (d) disintegrating agents, such as for example, agar-agar, calcium carbonate, potato or tapioca starch, alginic acid certain complex silicates, or sodium carbonate; (e) solution retarders, such as for example, paraffin; (f) absorption accelerators, such as for example, quaternary ammonium compounds; (g) wetting agents, such as for example, cetyl alcohol or glycerol monostearate; (h) adsorbents, such as for example, kaolin or bentonite; and/or (i) lubricants, such as for example, talc, calcium stearate, magnesium stearate, solid polyethylene glycols, sodium lauryl sulfate, or mixtures thereof. In the case of capsules and tablets, the dosage forms may further comprise buffering agents.

In certain embodiments, solid dosage forms may be formulated as modified release and pulsatile release dosage forms containing excipients such as those detailed above for immediate release dosage forms together with additional excipients that act as release rate modifiers, these being coated on and/or included in the body of the device. Release rate modifiers include, but are not limited to, hydroxypropylmethyl cellulose, methyl cellulose, sodium carboxymethylcellulose, ethyl cellulose, cellulose acetate, polyethylene oxide, xanthan gum, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients.

In certain embodiments, the pharmaceutical compositions of the invention may further comprise fast dispersing or dissolving dosage formulations (FDDFs) containing the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e., where the drug substance is insoluble, a fast dispersing dosage form may be prepared, and where the drug substance is soluble, a fast dissolving dosage form may be prepared.

Solid compositions of a similar type may also be employed as fillers in soft or hard filled gelatin capsules using such excipients as lactose or milk sugar, as well as high molecular weight polyethylene glycols, and the like.

In certain embodiments, solid dosage forms such as tablets, dragees, capsules, and granules can be prepared with coatings and shells, such as enteric coatings and others well-known to one of ordinary skill in the art. They may also comprise opacifying agents, and can also be of such composition that they release the active compound(s) in a delayed, sustained, or controlled manner. Examples of embedding compositions that can be employed are polymeric substances and waxes. The active compound(s) can also be in micro-encapsulated form, if appropriate, with one or more of the above-mentioned excipients.

In certain embodiments, liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and/or emulsifiers, as for example, ethyl alcohol, isopropyl alcohol, ethyl carbonate, benzyl benzoate, propylene glycol, 1,3-butylene glycol, oils, in particular, cottonseed oil, groundnut oil, corn germ oil, olive oil, castor oil, or sesame seed oil, glycerol, tetrahydrofurfuryl alcohol, polyethylene glycols or fatty acid esters of sorbitan, or mixtures of these substances, and the like.

Besides such inert diluents, the pharmaceutical composition can also include adjuvants, such as wetting agents, emulsifying and suspending agents, sweetening, flavoring, and perfuming agents. The pharmaceutical composition may further include suspending agents, such as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

In certain embodiments, pharmaceutical compositions of the present invention may also be configured for treatments in veterinary use, where a compound of the present invention, or a veterinarily acceptable salt thereof, or veterinarily acceptable solvate or pro-drug thereof, is administered as a suitably acceptable formulation in accordance with normal veterinary practice and the veterinary practitioner will determine the dosing regimen and route of administration which will be most appropriate for a particular animal.

If a combination of active agents is administered, then they may be administered simultaneously, separately or sequentially.

Compounds of Formula (I) may be prepared by a variety of synthetic routes. Representative preparation procedures are outlined below. Unless otherwise indicated, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰,

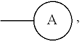

X and Y are as defined herein above. P represents a protecting group. In a desired reaction step of the processes described hereafter, NH or hydroxyl protections and removal of the protecting groups used may be carried out according to the known procedures such as those described in Protective Groups in Organic Synthesis edited by T. W. Greene et al. (John Wiley &Sons, 1991). Isolated hydroxyl groups can generally be protected as ethers, acetals and esters. In general, benzyl-type protecting group are removed by hydrogenolysis, silyl ethers by reaction with fluoride ions or under slightly acidic conditions and several 2-substituted ethyl ethers can be cleaved by beta-elimination reactions. It is to be understood that the present invention is not limited to the specific details of the Examples provided below. In this discussion which follows, certain common chemical and procedural abbreviations and acronyms therefore have been employed which include: Me (methyl); Et (ethyl); EtOAc (ethyl acetate); Bn (benzyl); THF (tetrahydrofuran); DMF (dimethylformamide); Boc (tert-butyloxycarbonyl); DMAP (1,1′-dimethylaminopyridine); DIBAL (diisobutylaluminum hydride); eq (equivalent); RP (reverse phase); HPLC (high performance liquid chromatography); TLC (thin layer chromatography). MOM(methoxymethyl); The compounds of formula (I) is most conveniently synthesized by employing procedures analogous to those known in the chemical arts for the production of similar compounds. Exemplary processes for the manufacture of compounds of formula (I) as defined in detail hereinabove are provided as further features of the invention and are illustrated by the following procedures. Examples of methods of preparing compounds of the present invention as described herein are provided by Scheme 1, 2, 3 and 4 below and the description that follows.

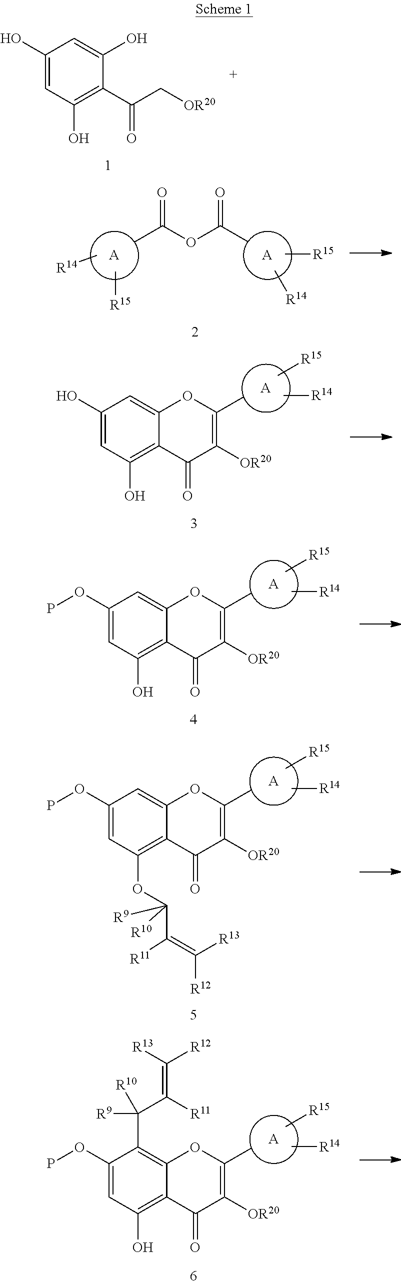

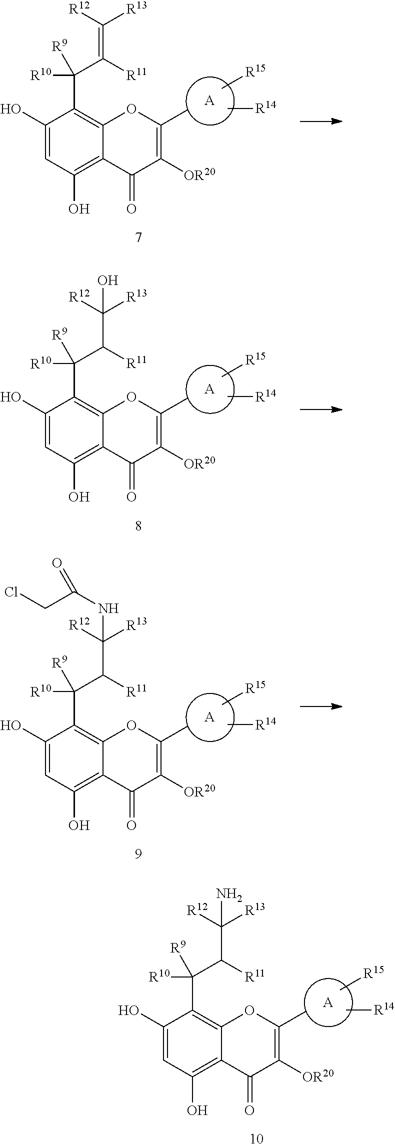

Referring to Scheme 1 above, a compound of the formula (I) may be prepared through several steps. A compound of formula 3 maybe prepared by condensation of the compound of formula 1 with the compound of formula 2 in a reaction inert solvent. Suitable solvents used in this reaction include ethers such as DME (1,2-dimethoxyethane), 1,2-diethoxyethane; THF, DMF; N,N-dimethylacetamide and N-methyl-2-pyrrolidinone. Preferred solvents used in this reaction are 1,2-dimethoxyethane, 1,2-diethoxyethane. This reaction may be conducted in the presence of a stoichiometric or catalytic amount of add such as triethylamine, N-ethyl-N-isopropylpropan-2-amine. This reaction is generally carried out at a temperature from about 0° C. to about 140° C., preferably at the reflux temperature of the solvent for about 1 to about 20 hours.

A compound of formula 4 is prepared by protecting 7-hydroxyl group of compound 3 with ethers in inert reaction solvents. Suitable solvents used in this reaction include ethers such as DME (1,2-dimethoxyethane), 1,2-diethoxyethane; THF; DMF; N,N-dimethylacetamide and N-methyl-2-pyrrolidinone. Preferred solvent used in this reaction is DMF. This reaction may be conducted in the presence of a stoichiometric or catalytic amount of add such as triethylamine, N-ethyl-N-isopropylpropan-2-amine. This reaction is generally carried out at a temperature from about 0° C. to about 80° C. for about 1 to about 20 hours.

A compound of formula 5 may be prepared by reaction of a compound of formula 4 with a bromide in reaction inert solvents. Suitable solvents used in this reaction include ethers such as DME (1,2-dimethoxyethane), 1,2-diethoxyethane; THF; DMF; N,N-dimethylacetamide and N-methyl-2-pyrrolidinone; CH₂Cl₂; CHCl₃. Preferred solvents used in this reaction are CH₂Cl₂. This reaction may be conducted in the presence of a stoichiometric or catalytic amount of add such as triethylamine, N-ethyl-N-isopropylpropan-2-amine, tetrabutylammonium hydroxide. Preferred base used in this reaction is tetrabutylammonium hydroxide. This reaction is generally carried out at a temperature from about 0° C. to about 80° C. for about 1 to about 20 hours.

A compound of formula 6 may be prepared by heating a compound of formula 5 in reaction inert solvents. Suitable solvents used in this reaction include ethers DMF; N,N-dimethylacetamide and N-methyl-2-pyrrolidinone; N,N-diethylaniline; N,N-dimethylaniline. Preferred solvent used in this reaction is N,N-diethylaniline. This reaction is generally carried out at a temperature from about 50° C. to about 300° C. for about 1 to about 20 hours. The preferred reaction temperature is from about 200° C. to about 300° C. for about 1 to about 20 hours.

A compound of formula 7 may be prepared by de-protecting the protective group of a compound of formula 6 in reaction inert solvents. Suitable solvents used in this reaction include ethers such as DME (1,2-dimethoxyethane), 1,2-diethoxyethane; dioxane; alcohols such as methanol, ethanol, isopropanol; THF; DMF; N,N-dimethylacetamide and N-methyl-2-pyrrolidinone; CH₂Cl₂; CHCl₃. Preferred solvent used in this reaction is isopropanol. This reaction is generally carried out at a temperature from about 0° C. to about 150° C. for about 10 minutes to about 20 hours. The preferred reaction temperature is from about 10° C. to about 80° C. for about 30 minutes to about 4 hours.

A compound of formula 8 may be prepared by reaction of a compound of formula 7 with water under acidic conditions in reaction inert solvents. Suitable solvents used in this include ethers such as DME (1,2-dimethoxyethane), 1,2-diethoxyethane; dioxane; alcohols such as methanol, ethanol, isopropanol; acetone; THF; DMF; N,N-dimethylacetamide and N-methyl-2-pyrrolidinone; or a mixture of above mentioned solvents with water. Preferred solvents used in this reaction are acetone-water (1:1 v/v). This reaction is generally carried out at a temperature from about 0° C. to about 150° C. for about 10 minutes to about 20 hours. The preferred reaction temperature is from about 50° C. to about 100° C. for about 2 minutes to about 8 hours.

A compound of formula 9 may be prepared by reaction of a compound of formula 8 with 2-chloroacetonitril. The reaction may carried out without solvents or with solvents include ethers such as DME (1,2-dimethoxyethane), 1,2-diethoxyethane; dioxane; THF; DMF; N,N-dimethylacetamide and N-methyl-2-pyrrolidinone. This reaction is generally carried out at a temperature from about −50° C. to about 50° C. for about 1 hour to about 20 hours. The preferred reaction temperature is from about −20° C. to about 50° C. for about 2 hours to about 8 hours.

A compound of formula 10 may be prepared by reaction of a compound of formula 9 with thiourea under acidic conditions in reaction inert solvents. Suitable solvents used in this include ethers such as DME (1,2-dimethoxyethane), 1,2-diethoxyethane; dioxane; alcohols such as methanol, ethanol, isopropanol; acetone; THF; DMF; N,N-dimethylacetamide and N-methyl-2-pyrrolidinone; This reaction is generally carried out at a temperature from about 0° C. to about 200° C. for about 1 hour to about 100 hours. The preferred reaction temperature is from about 60° C. to about 150° C. for about 30 hours to about 50 hours.

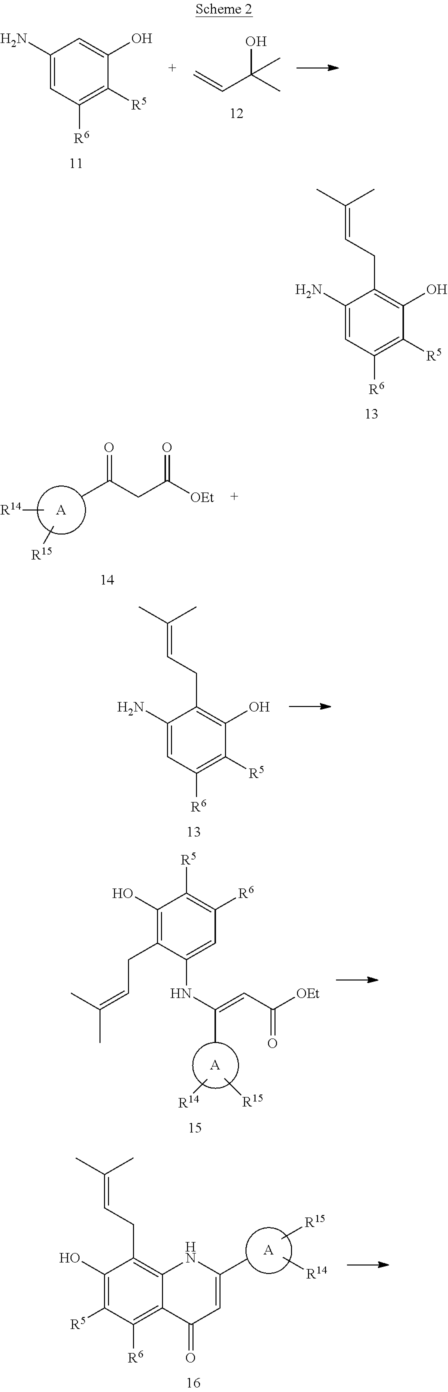

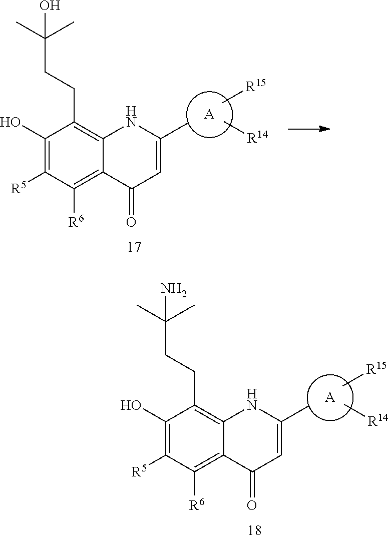

Referring to scheme 2, wherein R⁵, R⁶, R¹⁴, R¹⁵ and

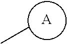

are defined above, compounds of Formula 13, can be prepared from the corresponding Formula 11 and Formula 12 compounds. Generally, a mixture of Formula 11 compound and Formula 12 compound in an aqueous acidic solution, such as citric acid solution, is heated to at a temperature of about ambient temperature to about 100° C., preferably at reflux temperature of the solvent for about one hour to about ten hours, preferably four to six hours.

Compounds of formula 15, can be prepared from the corresponding Formula 13 and Formula 14 compounds. A compound of formula 15 may be prepared by reaction of a compound of formula 13 with a compound of formula 14 under acidic conditions, such as 4-methylbenzenesulfonic acid in reaction inert solvents. Suitable solvents used in this include ethers such as toluene, DME (1,2-dimethoxyethane), 1,2-diethoxyethane; dioxane; THF; DMF; N,N-dimethylacetamide and N-methyl-2-pyrrolidinone, preferably toluene; This reaction is generally carried out at a temperature from about 60° C. to about 200° C. for about 1 hour to about 100 hours. The preferred reaction temperature is from about 90° C. to about 120° C. for about 10 hours to about 30 hours.

A compound of formula 16 may be prepared by heating a compound of formula 15 in reaction inert solvents. Suitable solvents used in this include ethers such as diphenyl ether, toluene, DME (1,2-dimethoxyethane), 1,2-diethoxyethane; dioxane; THF; DMF; N,N-dimethylacetamide and N-methyl-2-pyrrolidinone, preferably diphenyl ether; This reaction is generally carried out at a temperature from about 60° C. to about 200° C. for about 1 hour to about 20 hours. The preferred reaction temperature is from about 90° C. to about 150° C. for about 5 hours to about 15 hours.

A compound of formula 17 and 18 may be prepared by the similar procedures described in the preparation of compounds 8, 9 and 10 in Scheme 1.

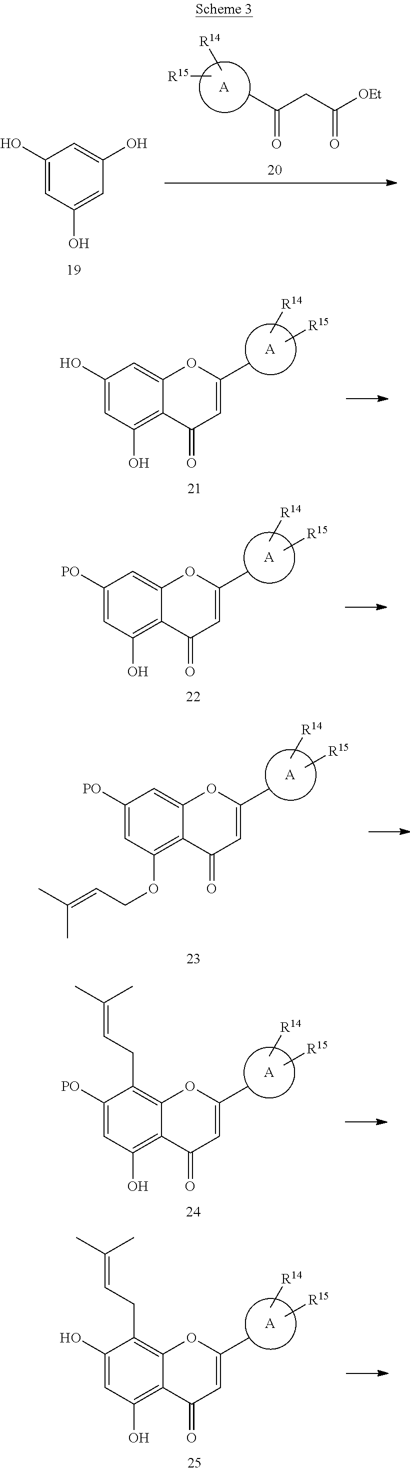

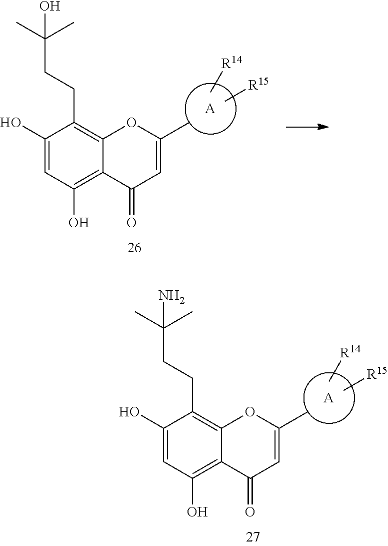

Referring to scheme 3, wherein P and

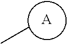

are defined as above compounds of Formula 21, can be prepared from the corresponding Formula 19 and Formula 20 compounds. Generally, a mixture of Formula 19 compound and Formula 20 compound is heated to at a temperature of about 150° C. to about 200° C. in microwave reactor to about 1 minute to 30 minutes.

A compound of formula 22 is prepared by protecting its 7-hydroxyl group of compound 21 with ethers in inert reaction solvents. Suitable solvents used in this reaction include ethers such as DME (1,2-dimethoxyethane), 1,2-diethoxyethane; THF; DMF; N,N-dimethylacetamide and N-methyl-2-pyrrolidinone. Preferred solvent used in this reaction is DMF. This reaction may be conducted in the presence of a stoichiometric or catalytic amount of add such as triethylamine, N-ethyl-N-isopropylpropan-2-amine. This reaction is generally carried out at a temperature from about 0° C. to about 80° C. for about 1 to about 20 hours.

A compound of formula 23 may be prepared by reaction of a compound of formula 22 with a bromide in reaction inert solvents. Suitable solvents used in this reaction include ethers such as DME (1,2-dimethoxyethane), 1,2-diethoxyethane; THF; DMF; N,N-dimethylacetamide and N-methyl-2-pyrrolidinone; CH₂Cl₂; CHCl₃ and toluene or a mixture of solvents mentioned above. Preferred solvents used in this reaction are a mixture of CH₂Cl₂ and toluene. This reaction may be conducted in the presence of a stoichiometric or catalytic amount of add such as triethylamine, N-ethyl-N-isopropylpropan-2-amine, tetrabutylammonium hydroxide. Preferred base used in this reaction is tetrabutylammonium hydroxide. This reaction is generally carried out at a temperature from about 0° C. to about 80° C. for about 1 to about 20 hours.

A compound of formula 24 may be prepared by heating a compound of formula 23 in reaction inert solvents. Suitable solvents used in this reaction include ethers DMF; N,N-dimethylacetamide and N-methyl-2-pyrrolidinone; N,N-diethylaniline; N,N-dimethylaniline. Preferred solvents used in this reaction are N,N-diethylaniline. This reaction is generally carried out at a temperature from about 50° C. to about 300° C. for about 1 to about 20 hours. The preferred reaction temperature is from about 200° C. to about 300° C. for about 1 to about 20 hours.

A compound of formula 25 may be prepared by de-protecting the protective group of a compound of formula 24 in reaction inert solvents. Suitable solvents used in this include ethers such as DME (1,2-dimethoxyethane), 1,2-diethoxyethane; dioxane; alcohols such as methanol, ethanol, isopropanol; THF; DMF; N,N-dimethylacetamide and N-methyl-2-pyrrolidinone; CH₂Cl₂; CHCl₃. This reaction is generally carried out at a temperature from about 0° C. to about 150° C. for about 10 minutes to about 20 hours. The preferred reaction temperature is from about 10° C. to about 80° C. for about 30 minutes to about 4 hours.

A compound of formula 26 and 27 may be prepared by the similar procedures described in the preparation of compounds 8, 9 and 10 in Scheme 1.

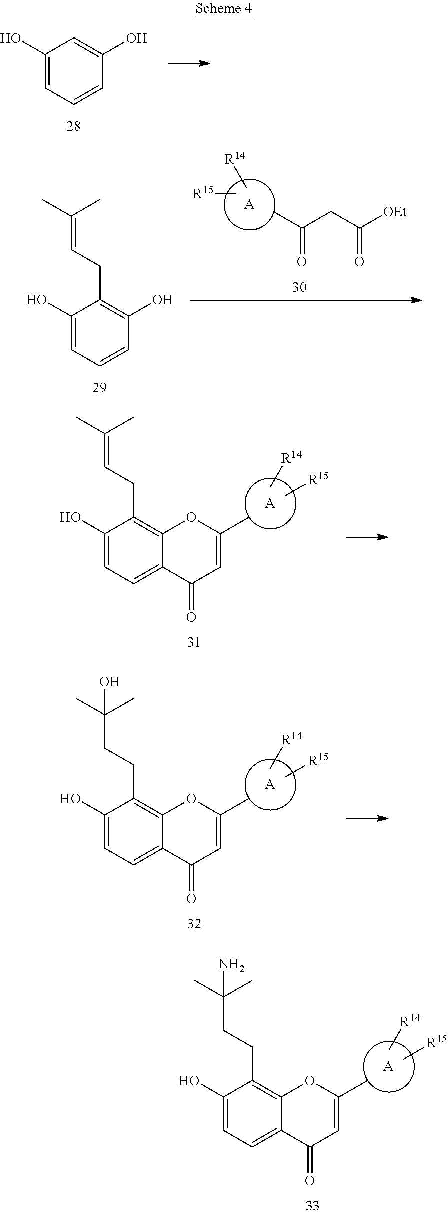

Referring to scheme 4, wherein R¹⁴, R¹⁵ and

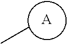

are defined as above, compounds of Formula 29 can be prepared from the corresponding Formula 28. Generally, a mixture of Formula 28 compound and prenyl bromide are heated to at a temperature of about 10° C. to about 100° C. about 2 hours to 30 hours in reaction inert solvents. Suitable solvents used in this include ethers such as DME (1,2-dimethoxyethane), 1,2-diethoxyethane; dioxane; The preferred reaction temperature is from about 1° C. to about 80° C. for about 5 hours to about 20 hours.

A compound of formula 31 may prepared from the corresponding Formula 29 and Formula 30 compounds. Generally, a mixture of Formula 29 compound and Formula 30 compound is heated to at a temperature of about 150° C. to about 300° C. in microwave reactor to about 1 minute to 60 minutes.

A compound of formula 32 and 33 may be prepared by the similar procedures described in the preparation of compounds 8, 9 and 10 in Scheme 1.

### EXAMPLES AND PREPARATIONS

The invention is illustrated in the following non-limiting examples in which, unless stated otherwise: room temperature or ambient temperature refer to the range of 18-250° C.; evaporation of solvent was carried out using a rotary evaporator under reduced pressure; reactions were monitored by thin layer chromatography (TLC) and reaction times are given for illustration only; melting points (m.p.) given are uncorrected (polymorphism may result in different melting points); structure and purity of all isolated compounds were assured by at least one of the following techniques: TLC, mass spectrometry, nuclear magnetic resonance (NMR), high pressure liquid chromatography (HPLC). Yields are given for illustrative purpose only.

### Preparation of 5,7-dihydroxy-3-methoxy-2-(3,4-dimethoxyphenyl)-4H-chromen-4-one (compound 1)

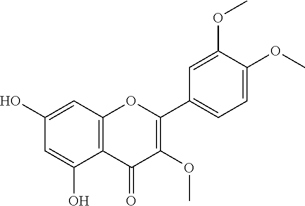

A mixture of 3,4-dimethoxybenzoic anhydride (26.0 g, 75 mmol), 1-(2,4,6-trihydroxyphenyl)-2-methoxyethanone (5.0 g, 25 mmol), NEt₃ (10 mL), 4A MS (10.0 g) and DME (40 mL) were refluxed for 10 h. After cooling to room temperature, Methanol (150 mL) containing KOH (9.2 g) was added and the mixture which resulted was refluxed for 2 h, water was added to the reaction mixture and the mixture which resulted was neutralized with HCl (6N) to pH about 8. The reaction mixture was extracted with EtOAc (100 mL×3). The organic extracts were combined, dried (Na₂SO₄). After removing the solvent the residue was purified by chromatography on silica gel to yield the title compound (6.5 g, 0.19 mol, yield 74%). ¹H NMR (400 MHz, DMSO-d₆): δ=12.64 (s, 1H), 10.86 (s, 1H), 7.69 (m, 2H), 7.62 (d, 1H, J=1.6 Hz), 7.15 (d, 1H, J=12.8 Hz), 6.49 (d, 1H, J=2.0 Hz), 6.22 (d, 1H, J=2.0 Hz), 3.86 (s, 6H), 3.81 (s, 3H).

### Preparation of 5-hydroxy-3-methoxy-7-(methoxymethoxy)-2-(3,4-dimethoxyphenyl)-4H-chromen-4-one (compound 2)

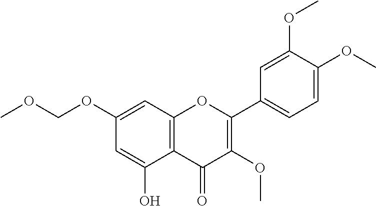

To a stirred solution of 5,7-dihydroxy-3-methoxy-2-(3,4-dimethoxyphenyl)-4H-chromen-4-one (17.0 g, 50 mmol) in dry DMF (140 mL) was added N,N-diisopropylethylamine (7.6 g, 59 mmol), followed by adding chloromethyl methyl ether (4.8 g, 60 mmol) to the mixture. The reaction mixture was allowed to stir for 4 h at room temperature. The reaction mixture was diluted with H₂O, adjusted to pH around 1 (1 N HCl). The reaction mixture was extracted with EtOAc. The EtOAc extracts were combined, washed with H₂O and concentrated under reduced pressure. The crude product was obtained after removing the solvent and was purified by chromatography on silica gel to give the title compound (7.0 g, 36%). ¹H NMR (400 MHz, CDCl₃) δ=12.60 (brs, 1H), 7.75 (dd, 1H, J₁=2.0 Hz, J₂=8.4 Hz), 7.70 (d, 1H, J=2.0 Hz), 7.00 (d, 1H, J=8.8 Hz), 6.63 (d, 1H, J=2.4 Hz), 6.47 (d, 1H, J=2.0 Hz), 5.25 (s, 2H), 3.98 (s, 3H), 3.97 (s, 3H), 3.87 (s, 3H), 3.51 (s, 3H); LCMS (ESI) m/z 389 [M+H]⁺.

### Preparation of 5-(3-methylbut-2-enyloxy)-3-methoxy-7-(methoxymethoxy)-2-(3,4-dimethoxyphenyl)-4H-chromen-4-one (compound 3)

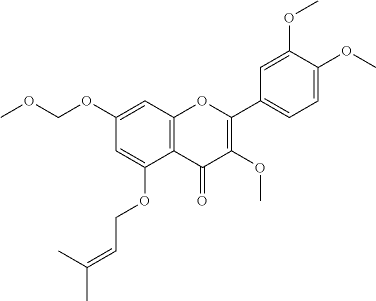

To a stirred solution of 5-hydroxy-3-methoxy-7-(methoxymethoxy)-2-(3,4-dimethoxyphenyl)-4H-chromen-4-one (5.0 g, 13 mmol) in CH₂Cl₂ (100 mL) was added tetrabutylammonium hydroxide (100 g, 33 mmol, 10% in water), followed by adding prenyl bromide (8.0 g, 53 mmol) to the above mixture and the reaction mixture was allowed to stir for 3 h at room temperature. The reaction mixture was diluted with H₂O. The reaction mixture was extracted with EtOAc. The EtOAc extracts were combined, washed with H₂O and concentrated under reduced pressure. The crude product was obtained after removing the solvent and was purified by chromatography on silica gel to give the title compound (5.5 g, 93%). ¹H NMR (400 MHz, CDCl₃) δ=7.71 (m, 2H), 6.97 (d, 1H, J=9.2 Hz), 6.71 (d, 1H, J=2.0 Hz), 6.44 (d, 1H, J=2.4 Hz), 5.60 (t, 1H, J=6.4 Hz), 5.26 (s, 1H), 4.70 (d, 1H, J=6.0 Hz), 3.97 (s, 3H), 3.96 (s, 3H), 3.87 (s, 3H), 3.52 (s, 3H), 1.77 (d, 6H, J=10.8 Hz); LCMS (ESI) m/z 457 [M+H]⁺.

### Preparation of 5-hydroxy-3-methoxy-7-(methoxymethoxy)-2-(3,4-dimethoxyphenyl)-8-(3-methylbut-2-enyl)-4H-chromen-4-one (compound 4)

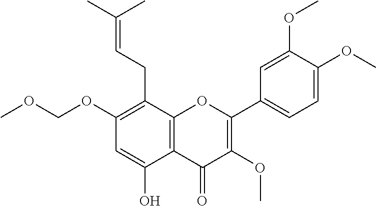

A mixture of 5-(3-methylbut-2-enyloxy)-3-methoxy-7-(methoxymethoxy)-2-(3,4-dimethoxyphenyl)-4H-chromen-4-one (2.3 g, 5.0 mmol) and N,N-diethylaniline (100 mL) was heated at 217° C. for 3 h with stirring. After cooling to room temperature the reaction mixture was diluted with H₂O and acidified (pH 1, 1 N HCl). The mixture was then extracted with EtOAc. The EtOAc extracts were combined and washed with H₂O. The solvent was evaporated under reduced pressure and the residue was purified by chromatography on silica gel to give the title compound (1.75 g, 76%). ¹H NMR (400 MHz, DMSO-d₆) δ=12.61 (s, 1H), 7.71 (dd, 1H, J₁=2.0 Hz, J₂=8.6 Hz), 7.66 (d, 1H, J=2.0 Hz), 7.19 (d, 1H, J=8.6 Hz), 6.58 (s, 1H), 5.36 (s, 2H), 5.22 (t, 1H, J=7.0 Hz), 3.87 (s, 3H), 3.84 (s, 3H), 3.83 (s, 3H), 3.49 (d, 2H, J=6.8 Hz), 3.40 (s, 3H), 1.77 (d, 6H, J=5.6 Hz); LCMS (ESI) m/z 457 [M+H]⁺.

### Preparation of 5,7-dihydroxy-3-methoxy-2-(3,4-dimethoxyphenyl)-8-(3-methylbut-2-enyl)-4H-chromen-4-one (compound 5)

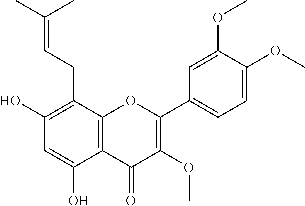

A mixture of 5-hydroxy-3-methoxy-7-(methoxymethoxy)-2-(3,4-dimethoxyphenyl)-8-(3-methylbut-2-enyl)-4H-chromen-4-one (0.91 g, 2.0 mmol), 4 N HCl (10 mL) and isopropanol (30 mL) was heated at 65° C. for 1 h. After cooling to room temperature, the mixture was extracted with EtOAc. The EtOAc extract was washed with H₂O, dried with Na₂SO₄ and evaporated under reduced pressure. The residue obtained was purified by chromatography on silica gel to give the title compound (0.72 g, 87%). ¹H NMR (400 MHz, acetone-d₆) δ=12.69 (s, 1H), 9.59 (s, 1H), 7.78 (dd, 1H, J₁=2.0 Hz, J₂=8.5 Hz), 7.74 (d, 1H, J=2.0 Hz), 7.13 (d, 1H, J=8.5 Hz), 6.33 (s, 1H), 5.30 (t, 1H, J=6.8 Hz), 3.91 (s, 3H), 3.90 (s, 6H), 3.52 (d, 1H, J=6.8 Hz), 1.60 (d, 6H, J=48 Hz); LCMS (ESI) m/z 413 [M+H]⁺.

### Preparation of 5,7-dihydroxy-8-(3-hydroxy-3-methylbutyl)-3-methoxy-2-(3,4-dimethoxyphenyl)-4H-chromen-4-one (compound 6)

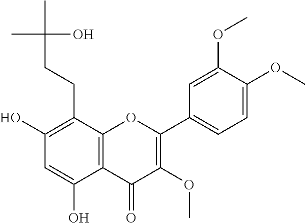

A mixture of 5,7-dihydroxy-3-methoxy-2-(3,4-dimethoxyphenyl)-8-(3-methylbut-2-enyl)-4H-chromen-4-one (91 mg, 0.2 mmol), 5% H₂SO₄ (5 mL) and acetone (20 mL) was heated at 65° C. for 5 h. After cooling to room temperature, the mixture was extracted with EtOAc. The EtOAc extracts were combined, washed with H₂O, dried with Na₂SO₄ and evaporated under reduced pressure. The residue was then purified by chromatography on silica gel to give the title compound (10 mg, 12%). ¹H NMR (400 MHz, DMSO-d₆) δ=12.57 (s, 1H), 10.71 (s, 1H), 7.74 (d, 1H, J=8.4 Hz), 7.67 (s, 1H), 7.11 (d, 1H, J=8.4 Hz), 6.29 (s, 1H), 4.27 (brs, 1H), 3.84 (s, 6H), 3.80 (s, 3H), 2.76 (m, 2H), 1.54 (m, 2H), 1.15 (s, 6H); LCMS (ESI) m/z 431 [M+H]⁺.

### Preparation of 2-chloro-N-(4-(3,5,7-trihydroxy-2-(4-methoxyphenyl)-4-oxo-4H-chromen-8-yl)-2-methylbutan-2-yl)acetamide (compound 7)

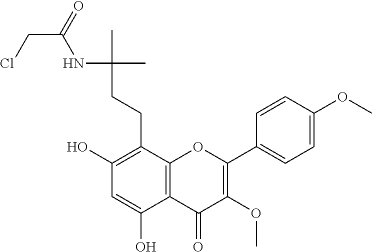

Glacial acetic acid (1.7 mL, 29 mmol) was added dropwise to a solution of 3,5,7-trihydroxy-8-(3-hydroxy-3-methylbutyl)-2-(4-methoxyphenyl)-4H-chromen-4-one (0.94 g, 2.4 mmol) in 2-chloroacetonitrile (49 mL, 780 mmol). The resulting solution was cooled to −15° C. and concentrated sulfuric acid (1.7 mL, 31 mmol) was added dropwise. The reaction mixture was stirred for 4 h under 20° C. The reaction mixture was then poured into ice and the mixture which resulted was basified with saturated aqueous NaHCO₃ and extracted with EtOAc. The organic layer was dried over Na₂SO₄ and concentrated under reduced pressure to give the crude product, which was purified by silica column chromatography (c-hexane/EtOAc, 3:1) afforded the tile compound (650 mg, 58%) as a yellow solid. ¹H NMR (500 MHz, DMSO-d₆) δ 1.35 (6H, s), 1.86-1.89 (2H, m), 2.68-2.71 (2H, m), 3.85 (3H, s), 4.02 (2H, s), 6.29 (1H, s), 7.12-7.15 (2H, m), 7.73 (1H, s), 8.17-8.19 (2H, m), 9.47 (1H, s), 10.68 (1H, s), 12.38 (1H, s); ¹³C NMR (125 MHz, DMSO-d₆) δ 17.12, 26.22(2), 42.57, 43.44, 53.22, 55.36, 97.79, 102.99, 106.22, 114.08(2C), 123.56, 129.31(2), 135.85, 146.13, 153.43, 158.24, 160.46, 161.36, 165.02, 176.27; ESIMS 462 [M+H]⁺.

### Preparation of 8-(3-amino-3-methylbutyl)-3,5,7-trihydroxy-2-(4-methoxyphenyl)-4H-chromen-4-one (compound 8)

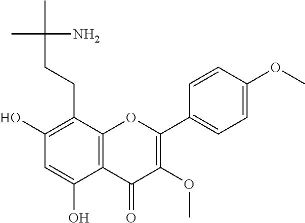

A solution of 2-chloro-N-(4-(3,5,7-trihydroxy-2-(4-methoxyphenyl)-4-oxo-4H-chromen-8-yl)-2-methylbutan-2-yl)acetamide (650 mg, 1.41 mmol), thiourea (130 mg, 1.69 mmol) and glacial acetic acid (1.4 mL) in EtOH (100 mL) was heated to reflux for 50 h. The reaction mixture was then cooled to room temperature and filtered. The filtrate was concentrated, basified using saturated aqueous NaHCO₃ and extracted with EtOAc. The combined organic solution was washed with brine, dried over Na₂SO₄ and concentrated to give the crude product, which was purified by chromatography on silica gel (CH₂Cl₂/EtOH, 4:1) afforded the title compound (210 mg, 37%) as a yellow solid. ¹H NMR (500 MHz, DMSO-d₆) δ 1.37 (6H, s), 1.73-1.77 (2H, m), 2.75-2.78 (2H, m), 3.85 (3H, s), 6.39 (1H, s), 7.12-7.13 (2H, m), 8.14-8.16 (2H, m), 9.51 (1H, s), 10.97 (1H, s), 12.38 (1H, s); ¹³C NMR (125 MHz, DMSO-d₆) δ16.76, 24.43(2), 53.50, 55.41, 55.99, 97.97, 102.92, 105.08, 114.05(2), 123.56, 129.27(2), 135.99, 146.08, 153.43, 158.48, 160.46, 161.77, 176.26; ESIMS 386 [M+H]⁺.

### Preparation the HCl salt of 8-(3-amino-3-methylbutyl)-3,5,7-trihydroxy-2-(4-methoxyphenyl)-4H-chromen-4-one (compound 9)

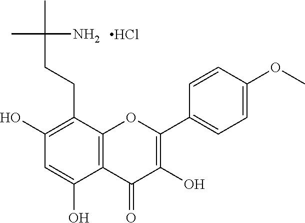

To a suspension of the 8-(3-amino-3-methylbutyl)-3,5,7-trihydroxy-2-(4-methoxyphenyl)-4H-chromen-4-one (210 mg, 0.55 mmol) in anhydrous EtOH (20 mL), a solution of concentrated hydrochloric acid (0.8 mL, 9.6 mmol) in anhydrous EtOH (5 mL) was added dropwise. The reaction mixture was stirred for 30 min, concentrated under reduced pressure to give the title product as a yellow solid. ¹H NMR (500 MHz, DMSO-d₆) δ 1.37 (6H, s), 1.72-1.75 (2H, m), 2.76-2.79 (2H, m), 3.86 (3H, s), 6.35 (1H, s), 7.12-7.13 (2H, m), 7.90 (3H, brs), 8.14-8.16 (2H, m), 9.54 (1H, s), 10.89 (1H, s), 12.38 (1H, s); ¹³C NMR (125 MHz, DMSO-d₆) δ 16.71, 24.41(2), 53.61, 55.42, 97.81, 103.06, 105.01, 114.06(2), 123.51, 129.27(2), 135.95, 146.16, 153.44, 158.54, 160.51, 161.37, 176.26; ESIMS 386 [M−HCl+H]⁺.

### Preparation of 2-(4-chlorophenyl)-5,7-dihydroxy-3-methoxy-4H-chromen-4-one (compound 10)

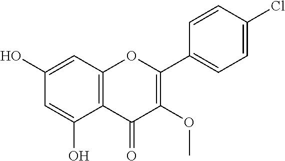

A mixture of 4-chlorobenzoic anhydride (31.3 g, 106 mmol), 1-(2,4,6-trihydroxyphenyl)-2-methoxyethanone (7.0 g, 35 mmol), NEt₃ (10 mL), 4A MS (10.0 g) and DME (70 mL) was refluxed for 10 h. After cooling to room temperature, methanol (150 mL) containing KOH (9.2 g) was added to the reaction mixture and then the reaction mixture was refluxed for 2 h. After cooling to room temperature, water was added and the mixture was neutralized with HCl (6N) to pH 8. The mixture was extracted with EtOAc (100 mL×3), dried and evaporated. The crude product was then purified by chromatography on silica gel to provide the title compound (4.3 g, 38%). LCMS (ESI) m/z 319.7 (M+H)⁺.

### Preparation of 2-(4-chlorophenyl)-5-hydroxy-3-methoxy-7-(methoxymethoxy)-4H-chromen-4-one (compound 11)

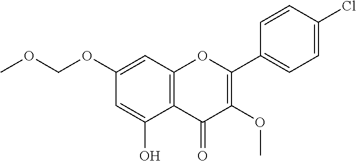

To a stirred solution of 2-(4-chlorophenyl)-5,7-dihydroxy-3-methoxy-4H-chromen-4-one (4.3 g, 13.3 mmol,) in dry DMF (40 mL) was added N,N-diisopropylethylamine (2.1 g, 16 mmol), followed by adding chloromethyl methyl ether (1.3 g, 15.8 mmol) to the above mixture. The reaction mixture was allowed to stir for 4 h at room temperature. The reaction mixture was diluted with H₂O, adjusted to pH 1 (1 N HCl), and extracted with EtOAc. The EtOAc extract was washed with H₂O, concentrated under reduced pressure. The crude product was then purified by chromatography on silica gel to give title compound (3.80 g, 79%). LCMS (ESI) m/z 363.8 [M+H]⁺.

### Preparation of 5-(3-methylbut-2-enyloxy)-2-(4-chlorophenyl)-3-methoxy-7-(methoxymethoxy)-4H-chromen-4-one (compound 12)

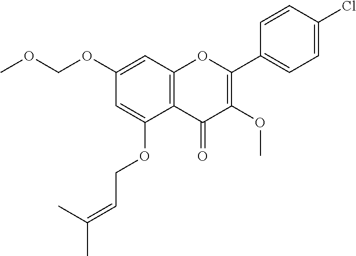

To a stirred solution of 2-(4-chlorophenyl)-5-hydroxy-3-methoxy-7-(methoxymethoxy)-4H-chromen-4-one (3.80 g, 10.5 mmol) in CH₂Cl₂ (100 mL) was added tetrabutylammonium hydroxide (80.0 g, 26.0 mmol, 10% in water), followed by adding prenyl bromide (6.3 g, 42 mmol). the reaction mixture was then allowed to stir 3 h at room temperature. The mixture was then diluted with H₂O and extracted with EtOAc. The extracts were combined and dried. The crude product was purified by chromatography on silica gel to give title compound (3.83 g, 85%). LCMS (ESI) m/z 431.4 [M+H]⁺.

### Preparation of 2-(4-chlorophenyl)-5-hydroxy-3-methoxy-7-(methoxymethoxy)-8-(3-methylbut-2-enyl)-4H-chromen-4-one (compound 13)

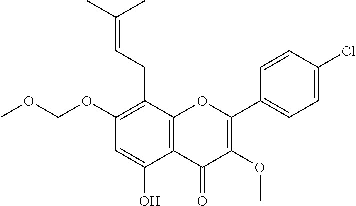

A mixture of 5-(3-methylbut-2-enyloxy)-2-(4-chlorophenyl)-3-methoxy-7-(methoxymethoxy)-4H-chromen-4-one (1.60 g, 3.7 mmol) and N,N-diethylaniline (70 mL) was slowly heated to 217° C. and stirred for 3 h. After cooling to room temperature, the reaction mixture was diluted with H₂O, acidified (pH 1, 1 N HCl), and extracted with EtOAc. The EtOAc extracts were combined, washed with H₂O and evaporated under reduced pressure, and the residue was purified by chromatography on silica gel to give title compound (0.80 g, 50%). ¹H NMR (400 MHz, acetone-d₆) δ=12.61 (s, 1H), 8.15 (d, 2H, J=8.8 Hz), 7.65 (d, 2H, J=8.8 Hz), 6.61 (s, 1H), 5.40 (s, 2H), 5.22 (t, 1H, J=6.8 Hz), 3.93 (s, 3H), 3.55 (d, 2H, J=6.8 Hz), 3.49 (s, 3H), 1.79 (s, 3H), 1.66 (s, 3H); LCMS (ESI) m/z 431.8 [M+H]⁺.

### Preparation of 2-(4-chlorophenyl)-5,7-dihydroxy-3-methoxy-8-(3-methylbut-2-enyl)-4H-chromen-4-one (compound 14)

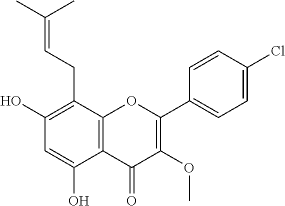

A mixture of 2-(4-chlorophenyl)-5-hydroxy-3-methoxy-7-(methoxymethoxy)-8-(3-methylbut-2-enyl)-4H-chromen-4-one (0.80 g, 1.85 mmol), 4 N HCl (5 mL) and isopropanol (30 mL) was heated at 50° C. for 2 h. After cooling to room temperature, the reaction mixture was extracted with EtOAc. The EtOAc extract was washed with H₂O, dried with Na₂SO₄ and evaporated under reduced pressure. The residue was purified by chromatography on silica gel to give title compound (0.58 g, 81%). ¹H NMR (400 MHz, acetone-d₆) δ=12.59 (s, 1H), 9.69 (s, 1H), 8.15 (d, 2H, J=8.8 Hz), 7.65 (d, 2H, J=8.8 Hz), 6.38 (s, 1H), 5.25 (t, 1H, J=6.8 Hz), 3.93 (s, 3H), 3.52 (d, 2H, J=6.8 Hz), 1.77 (s, 3H), 1.66 (s, 3H); LCMS (ESI) m/z 384.9 [M−H]⁻.

### Preparation of 3-amino-2-(3-methylbut-2-enyl)phenol (compound 15)

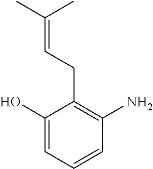

A mixture of m-aminophenol (5.45 g, 50 mmol), 2-methylbut-3-en-2-ol (4.30 g, 50 mmol) and aquoes citric acid (5%, 50 mL) was heated at 100° C. for 6 h, after cooling to room temperature, the mixture was washed with saturated NaHCO₃, dried over Na₂SO₄ and concentrated in vacuo. Then the crud product was purified by chromatography on silica gel and crystallized from petroleum ether and ethyl acetate (v:v=3:1) to afford the title compound in 10% yield (0.87 g). ¹H-NMR (400 MHz, CDCl₃): δ=6.89 (t, 1H, J=8.0 Hz), 6.31 (d, 1H, J=8.0 Hz), 6.25 (d, 1H, J=8.0 Hz), 5.16 (t, 1H, J=6.8 Hz), 4.81 (brs, 1H), 3.68 (brs, 2H), 3.31 (d, 1H, J=6.8 Hz), 1.78 (d, 6H, J=32 Hz); LCMS (ESI) m/z 178.1 (M+H)⁺.

### Preparation of ethyl (Z)-ethyl 3-(3-hydroxy-2-(3-methylbut-2-enyl)phenylamino)-3-(4-methoxyphenyl)acrylate (compound 16)

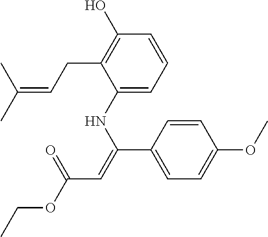

A mixture of 3-amino-2-(3-methylbut-2-enyl)phenol (0.87 g, 5.0 mmol), ethyl 3-(4-methoxyphenyl)-3-oxopropanoate (0.67 g, 3.0 mmol) and TsOH (0.05 g, 0.3 mmol) in toluene (10 mL) was heated to reflux for 24 h. After cooling to room temperature, dichloromethane (30 mL) was added to the reaction mixture. The organic layer was washed with water (10 mL×3) and dried with Na₂SO₄, concentrated. The crude product was purified by chromatography on silica gel using petroleum ether and ethyl acetate (v:v=20:1) to afford title compound with 40% yield (0.42 g); LCMS (ESI) m/z 382.2 (M+H)⁺.

### Preparation of 7-hydroxy-2-(4-methoxyphenyl)-8-(3-methylbut-2-enyl)quinolin-4(1H)-one (compound 17)

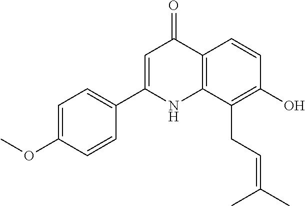

Ethyl 3-(3-hydroxy-2-(3-methylbut-2-enyl)phenylamino)-3-phenylacrylate (0.42 g, 1.20 mmol) was dissolved in diphenyl ether (20 mL) and heated to reflux for 5 h. The solvent was removed and the crude product was purified by chromatography on silica gel to afford the title compound (0.20 g, 54%). ¹H-NMR (CDCl₃): δ=8.04 (d, 1H, J=8.8 Hz), 7.55 (d, 2H, J=8.4 Hz), 7.04 (d, 2H, J=8.4 Hz), 6.96 (d, 1H, J=8.8 Hz), 6.49 (s, 1H), 5.30 (t, 1H, J=6.4 Hz), 3.90 (s, 3H), 3.71 (d, 2H, J=6.4 Hz), 3.70 (brs, 1H), 1.82 (d, 6H, J=34 Hz); LCMS (ESI) m/z 336.2 (M+H)⁺.

### Preparation of 2-(4-chlorophenyl)-5,7-dihydroxy-4H-chromen-4-one (compound 18)

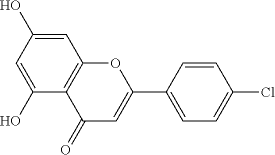

The mixture of methyl 3-(4-chlorophenyl)-3-oxopropanoate (10.6 g, 50 mmol) and 1,3,5-trihydroxylbenzene (8.1 g, 50 mmol) was reacted in micro wave reactor (170° C., run 1 min and hold 3 min) for three times. The medium was diluted with EtOAc and filtrated to obtain the title compound (3.5 g, 24.2%). LCMS (ESI): m/z 289 [M+H]⁺.

### Preparation of 2-(4-chlorophenyl)-5-hydroxy-7-(methoxymethoxy)-4H-chromen-4-one (compound 19)

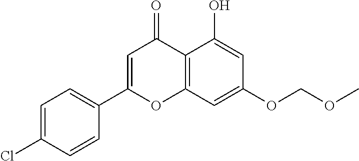

To a stirred solution of 2-(4-chlorophenyl)-5,7-dihydroxy-4H-chromen-4-one (8.8 g, 30.6 mmol) in dry DMF (80 mL) was added N,N-diisopropylethylamine (4.7 g, 36.7 mmol), followed by adding chloromethyl methyl ether to the mixture (2.95 g, 36.7 mmol). The reaction mixture was stirred at room temperature for 4 h. The reaction mixture was then poured into water (400 ml) and filtrated to provide the crude product which was used directly in next step without further purification. ¹HNMR (400M, acetone-d₆) δ=12.78 (s, 1H), 8.14 (d, J=8.8, 2H), 7.65 (d, J=8.8, 2H), 6.89 (s, 1H), 6.82 (d, J=2.0 Hz, 1H), 6.45 (d, J=2.0 Hz, 1H), 5.36 (s, 1H), 3.50 (s, 3H); LCMS (ESI): m/z 332 [M+H]⁺.

### Preparation of 5-(3-methylbut-2-enyloxy)-2-(4-chlorophenyl)-7-(methoxymethoxy)-4H-chromen-4-one (compound 20)

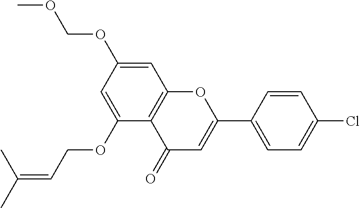

To a stirred solution of 2-(4-chlorophenyl)-5-hydroxy-7-(methoxymethoxy)-4H-chromen-4-one (10.0 g, 30 mmol) in DCM (100 mL) and toluene (100 mL) was added tetrabutylammonium hydroxide (153 g, 33 mmol, 10% water solution), followed by adding prenyl bromide (8.74 g, 60 mmol, 2 eq) to the reaction mixture and the reaction mixture was allowed to stir for 3 h at room temperature. The reaction mixture was diluted with H₂O (100 mL), extracted with EtOAc. The crude product was obtained after removing the solvent and purified by chromatography on silica gel to give title compound (8.0 g, 67%). ¹HNMR (400M, acetone-d₆) δ=8.05 (d, J=8.4, 2H), 7.60 (d, J=8.4, 2H), 6.87 (s, 1H), 6.63 (s, 1H), 6.60 (s, 1H), 5.53 (t, J=6.8, 1H), 5.35 (s, 1H), 4.70, (d, J=6.8, 2H) 3.50 (s, 3H), 1.79 (s, 3H), 1.78 (s, 3H); LCMS (ESI): m/z 401 [M+H]⁺.

### Preparation of 2-(4-chlorophenyl)-5-hydroxy-7-(methoxymethoxy)-8-(3-methylbut-2-enyl)-4H-chromen-4-one (compound 21)

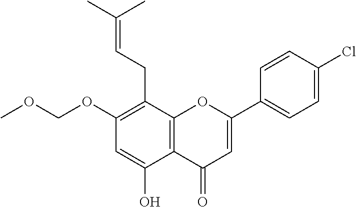

A mixture of 5-(3-methylbut-2-enyloxy)-2-(4-chlorophenyl)-7-(methoxymethoxy)-4H-chromen-4-one (4.5 g, 11.2 mmol) and N,N-diethylaniline (400 mL) was heated at 217° C. for 3 h with stirring. After cooling to room temperature, the reaction mixture was poured into the diluted hydrochloric acid solution. The precipitate was collected by filtration and then crystallized in EtOAc and Petroleum Ether (v/v=1:1) to give the title compound (2.0 g, 44.6%). ¹HNMR (400M, acetone-d₆) δ=12.81 (s, 1H), 8.10 (d, J=8.8, 2H), 7.66 (d, J=8.8, 2H), 6.85 (s, 1H), 6.60 (s, 1H), 5.40 (s, 2H), 5.27 (t, J=6.8, 1H), 3.59, (d, J=6.8, 2H), 3.50 (s, 3H), 1.84 (s, 3H), 1.68 (s, 3H); LCMS (ESI): m/z 401 [M+H]⁺.

### Preparation of 2-(4-chlorophenyl)-5,7-dihydroxy-8-(3-methylbut-2-enyl)-4H-chromen-4-one (compound 22)

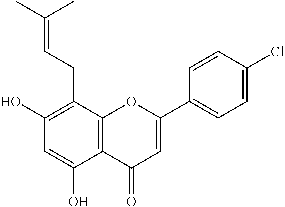

2-(4-chlorophenyl)-5-hydroxy-7-(methoxymethoxy)-8-(3-methylbut-2-enyl)-4H-chromen-4-one (0.5 g, 2.0 mmol) was dissolved in a mixture of CH₂Cl₂ (20 mL) and acetic acid (20 mL) at 20° C. and then seven drops of concentrate HCl was added to above mixture. The mixture which resulted was stirred at 20° C. for 18 h. The solid was collected by filtration which was rinsed with saturated NaHCO₃ solution to give the title compound as a yellow power (0.3 g, 42%). ¹HNMR (400M, DMSO-d₆) δ=12.71 (s, 1H), 11.04 (s, 1H), 8.05 (d, J=8.4, 2H), 7.66 (d, J=8.4, 2H), 6.96 (s, 1H), 6.37 (s, 1H), 5.19 (t, J=6.8, 1H), 3.44, (d, J=6.8, 2H), 1.76 (s, 3H), 1.63 (s, 3H); LCMS (ESI): m/z 357 [M+H]⁺.

### Preparation of 2-(3-chloro-4-methoxyphenyl)-5,7-dihydroxy-4H-chromen-4-one (compound 23)

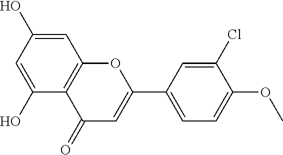

A mixture of ethyl 3-(3-chloro-4-methoxyphenyl)-3-oxopropanoate (12.2 g, 50 mmol) and 1,3,5-trihydroxylbenzene (8.1 g, 50 mmol) was reacted in micro wave reactor (160° C., run 1 min and hold 3 min) for three times. The reaction mixture was diluted with EtOAc and filtrated to obtain the title compound (3.2 g, 20.2%). ¹HNMR (400M, DMSO-d₆) δ=12.84 (s, 1H), 10.89 (brs, 1H, 8.15 (d, J=2.0 Hz, 1H), 8.04 (dd, J₁=2.0 Hz, J₂=8.8 Hz, 1H), 7.30 (d, J=8.8, 2H), 6.96 (s, 1H), 6.53 (d, J=2.0 Hz, 1H), 6.20 (d, J=2.0 Hz, 1H), 3.95 (s, 3H); LCMS (ESI): m/z 319 [M+H]⁺.

### Preparation of 2-(3-chloro-4-methoxyphenyl)-5-hydroxy-7-(methoxymethoxy)-4H-chromen-4-one (compound 24)

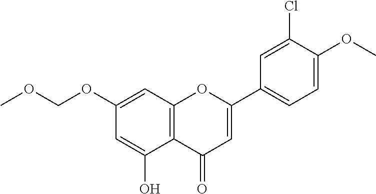

To a stirred solution of 2-(3-chloro-4-methoxyphenyl)-5,7-dihydroxy-4H-chromen-4-one (6.8 g, 21.5 mmol) in dry DMF (80 mL) was added N,N-diisopropylethylamine (3.3 g, 25.8 mmol), followed by adding chloromethyl methyl ether (2.10 g, 26.0 mmol) to the reaction mixture. The reaction mixture was stirred at room temperature for 4 h. The reaction mixture was poured into water (400 mL) and the precipitate was filtrated to furnish the crude product which was used directly in next step without further purification. ¹HNMR (400M, DMSO-d₆) δ=12.79 (s, 1H), 8.15 (d, J=2.0 Hz, 1H), 8.04 (dd, J₁=2.0 Hz, J₂=8.4 Hz, 1H), 7.27 (d, J=8.4, 1H), 7.00 (s, 1H), 6.86 (d, J=2.0 Hz, 1H), 6.41 (d, J=2.0 Hz, 1H), 5.31 (s, 2H), 3.95 (s, 3H), 3.41 (s, 3H); LCMS (ESI): m/z 363 [M+H]⁺.

### Preparation of 5-(3-methylbut-2-enyloxy)-2-(3-chloro-4-methoxyphenyl)-7-(methoxymethoxy)-4H-chromen-4-one (compound 25)

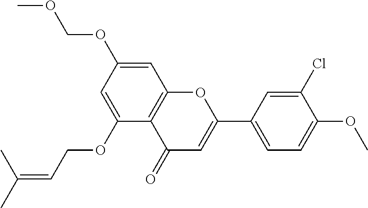

To a stirred solution of 2-(3-chloro-4-methoxyphenyl)-5-hydroxy-7-(methoxymethoxy)-4H-chromen-4-one (7.8 g, 21.5 mmol) in DCM (100 mL) and toluene (100 mL) was added tetrabutylammonium hydroxide (200 g, 43 mmol, 10% water solution), followed by adding prenyl bromide (12.5 g, 86 mmol, 2 eq) to the above mixture. The reaction mixture was allowed to stir for 3 h at room temperature. The reaction mixture was diluted with H₂O (100 mL) and extracted with EtOAc. The organic layers were combined, dried and evaporated. The crude product was then purified by chromatography on silica gel to give the title compound (4.5 g, 48%). ¹HNMR (400M, CDCl₃) δ=7.91 (d, J=2.0 Hz, 1H), 7.74 (dd, J₁=2.4 Hz, J₂=8.8 Hz, 1H), 7.03 (d, J=8.8 Hz, 1H), 6.77 (d, J=2.0 Hz, 1H), 6.57 (s, 1H), 6.47 (d, J=2.4 Hz, 1H), 5.58 (t, J=6.4 Hz, 1H), 5.27 (s, 2H), 4.67 (d, J=6.4 Hz, 1H), 3.98 (s, 3H), 3.54 (s, 3H); LCMS (ESI): m/z 431 [M+H]⁺.

### Preparation of 2-(3-chloro-4-methoxyphenyl)-5-hydroxy-7-(methoxymethoxy)-8-(3-methylbut-2-enyl)-4H-chromen-4-one (compound 26)

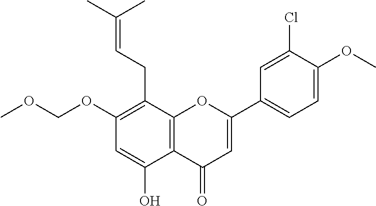

A mixture of 5-(3-methylbut-2-enyloxy)-2-(3-chloro-4-methoxyphenyl)-7-(methoxymethoxy)-4H-chromen-4-one (4.5 g, 10.4 mmol) and N,N-diethylaniline (400 mL) was heated at 217° C. for 3 h under stirring. After cooling to room temperature, the reaction mixture was poured into the diluted hydrochloric acid solution and the precipitate was filtrated and then crystallized in EtOAc and Petroleum Ether (v/v=1:1) to give the title compound (1.6 g, 35.6%). ¹HNMR (400M, acetone-d₆) δ=12.87 (s, 1H), 8.09 (s, 1H), 8.04 (d, J=8.8, 1H), 7.35 (d, J=8.8, 1H), 6.78 (s, 1H), 6.58 (s, 1H), 5.39 (s, 1H), 5.26 (t, J=6.8, 1H), 4.04 (s, 3H), 3.59 (d, J=6.4 Hz, 2H), 3.49 (s, 3H), 1.87 (s, 3H), 1.69 (s, 3H); LCMS (ESI): m/z 431 [M+H]⁺.

### Preparation of 2-(3-chloro-4-methoxyphenyl)-5,7-dihydroxy-8-(3-methylbut-2-enyl)-4H-chromen-4-one (compound 27)

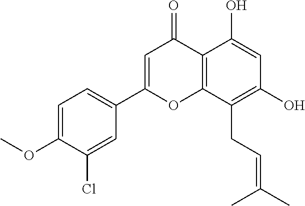

2-(3-chloro-4-methoxyphenyl)-5-hydroxy-7-(methoxymethoxy)-8-(3-methylbut-2-enyl)-4H-chromen-4-one (0.5 g, 2.0 mmol) was dissolved in a mixture of CH₂Cl₂ (20 mL) and acetic acid (20 mL) at 20° C. and concentrate HCl (0.15 mL) was added to above mixture. The mixture which resulted was stirred at 20° C. for 18 h. The solid was collected by filtration and rinsed with saturated NaHCO₃ solution to give the title compound as a yellow power (0.32 g, 41.5%). ¹HNMR (400M, acetone-d₆) δ=12.77 (s, 1H), 10.86 (s, 1H), 8.06 (s, 1H), 8.01 (d, J=8.8, 1H), 7.33 (d, J=8.8, 1H), 6.95 (s, 1H), 6.31 (s, 1H), 5.16 (t, J=6.4, 1H), 3.95 (s, 3H), 3.43 (d, J=6.4 Hz, 2H), 1.78 (s, 3H), 1.64 (s, 3H); LCMS (ESI): m/z 387 [M+H]⁺.

### Preparation of 2-(3-methylbut-2-enyl)benzene-1,3-diol (compound 28)

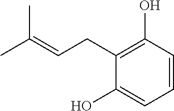

Metallic sodium (1.04 g, 45.22 mmol) was added portion wise to an ethereal solution (50 mL) of resorcinol (1.26 g, 11.24 mmol). After the mixture was stirred for 1.5 h, prenyl bromide (1.70 g, 11.24 mmol) was added drop-wise, then the reaction mixture was refluxed for 10 h. After removing the un-reacted sodium, the solution was acidified with 0.1 M aq. HCl (4 mL) and extracted with ether. The organic extract was washed with brine, dried over Na₂SO₄ and concentrated in vacuo to give the crude product, which was purified by silica column chromatography (hexane/EtOAc, 6:1) afforded the title compound (200 mg, 10%) as a yellow oil. ¹H NMR (500 MHz, CDCl₃) δ 1.76 (3H, brs), 1.83 (3H, brs), 3.42 (2H, d, J=7.0 Hz), 5.10 (2H, s), 5.27 (1H, t, J=7.0 Hz), 6.40 (2H, d, J=8.0 Hz), 6.94 (1H, t, J=8.5 Hz); ESIMS 177 [M−H]⁻.

### Preparation of 2-(4-(trifluoromethyl)phenyl)-7-hydroxy-8-(3-methylbut-2-enyl)-4H-chromen-4-one (compound 29)

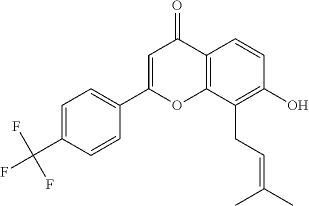

A mixture of ethyl 4-trifluoromethylbenzoyl acetate (420 mg, 1.46 mmol) and 2-(3-methylbut-2-enyl)benzene-1,3-diol (130 mg, 0.73 mmol) was irradiated with microwaves (Biotage, with the temperature control set to 240° C.) for 30 min. The crude product was purified by silica column chromatography (hexane/EtOAc, 1:2) afforded the title compound (90 mg, 40%) as a white solid. ¹H NMR (500 MHz, DMSO-d₆) δ 1.64 (3H, brs), 1.79 (3H, s), 3.60 (2H, d, J=7.0 Hz), 5.25 (1H, t, J=7.0 Hz), 7.02 (1H, d, J=9.0 Hz), 7.04 (1H, s), 7.79 (1H, d, J=9.0 Hz), 7.96 (2H, d, J=8.5 Hz), 8.26 (2H, d, J=8.0 Hz), 10.76 (1H, s); ESIMS 375 [M+H]⁺.

### Preparation of 7-hydroxy-8-(3-methylbut-2-enyl)-2-(4-methylphenyl)-4H-chromen-4-one (compound 30)

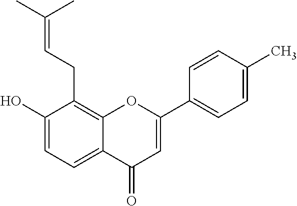

The title compound was synthesized by a similar procedure of preparation of compound 2-(4-(trifluoromethyl)phenyl)-7-hydroxy-8-(3-methylbut-2-enyl)-4H-chromen-4-one as a white solid (65 mg, 28%). ¹H NMR (500 MHz, DMSO-d₆) δ 1.64 (3H, s), 1.79 (3H, s), 2.40 (3H, s), 3.59 (2H, d, J=6.5 Hz), 5.28 (1H, m), 6.84 (1H, s), 6.99 (1H, d, J=9.0 Hz), 7.40 (2H, d, J=8.0 Hz), 7.76 (1H, d, J=9.0 Hz), 7.94 (2H, d, J=8.5 Hz), 10.68 (1H, s); ¹³C NMR (125 MHz, DMSO-d₆) δ17.9, 21.0, 21.9, 25.4, 105.7, 114.0, 115.1, 116.3, 121.9, 123.4, 126.0(2C), 128.8, 129.7(2C), 131.5, 141.7, 155.1, 159.8, 162.0, 176.7; ESIMS 321 [M+H]⁺.

### Preparation of 2-(4-fluorophenyl)-7-hydroxy-8-(3-methylbut-2-enyl)-4H-chromen-4-one (compound 31)

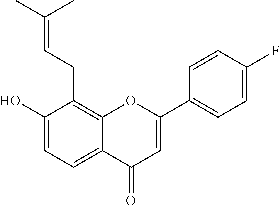

The title compound was synthesized by a similar procedure of preparation of compound 2-(4-(trifluoromethyl)phenyl)-7-hydroxy-8-(3-methylbut-2-enyl)-4H-chromen-4-one as a white solid (68 mg, 30%). ¹H NMR (500 MHz, DMSO-d₆) δ 1.64 (3H, s), 1.78 (3H, s), 3.59 (2H, d, J=7.0 Hz), 5.24 (1H, m), 6.90 (1H, s), 6.99 (1H, d, J=8.5 Hz), 7.43-7.46 (2H, m), 7.77 (1H, d, J=8.5 Hz), 8.09-8.12 (2H, m), 10.70 (1H, s); ¹³C NMR (125 MHz, DMSO-d₆) δ 17.9, 25.4, 106.3, 114.1, 115.1, 116.2 (d, J=21.5 Hz), 121.9, 123.5, 128.2, 128.7 (d, J=8.6 Hz), 131.6, 155.2, 159.9, 161.0, 163.9 (d, J=248.9 Hz), 176.7; ESIMS 325 [M+H]⁺.

### Preparation of 2-(3-(tetrahydro-2H-pyran-2-yloxy)phenoxy)-tetrahydro-2H-pyran (compound 32)

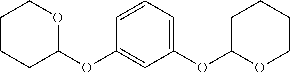

A mixture of resorcinol (11 g), DCM (100 mL) and PPTS (1 g) was stirred at reflux for 10 min, an 3,4-dihydro-2H-pyran (16.8 g) was added slowly, then the mixture was stirred at r.t for 3 h. The resulting solution was concentrated and purified by flash chromatography to afford title compound (22 g, 80%) as a white solid. ¹H NMR (300 MHz, CDCl₃): δ 7.20 (t, 1H), 6.85 (t, 3H), 5.40 (s, 2H), 3.60-4.00 (t, 4H), 1.60-2.10 (t, 12H); LCMS [M+H]⁺: 279; Purity (LCMS)>95%.

### Preparation of 2-(2-(3-methylbut-2-enyl)-3-(tetrahydro-2H-pyran-2-yloxy)phenoxy)-tetrahydro-2H-pyran (Compound 33)

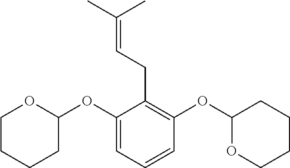

A solution of 2-(3-(tetrahydro-2H-pyran-2-yloxy)phenoxy)-tetrahydro-2H-pyran (12 g) in 200 mL of cyclohexane was added 17 mL of n-BuLi at 0° C. under N₂ atmosphere. The reaction mixture was refluxed for 3 h. After cooling, the 1-bromo-3-methylbut-2-ene (8 g) was added and the mixture resulted was refluxed for 3 h. After Cooling, the mixture was diluted with 200 mL of H₂O, extracted with EtOAc, purified by flash chromatography to afford the title compound (10 g, 60%) as an oil. ¹H NMR (300 MHz, CDCl₃): δ 6.70-7.20 (t, 3H), 5.45 (s, 2H), 5.25 (s, 1H), 3.60-4.00 (t, 4H), 3.45 (s, 1H), 1.80 (s, 6H), 1.60-2.10 (t, 12H); LCMS [M+H]⁺: 347; Purity (LCMS)>95%.

### Preparation of 2-(3-methylbut-2-enyl)benzene-1,3-diol (compound 34)

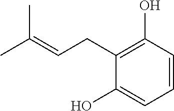

A mixture of Compound 33 (10 g), methanol (250 mL), oxalic acid (15 g) and water (40 mL) was stirred at room temperature for 1 h. The resulting mixture was concentrated in vacuum, purified by flash chromatography to afford title compound (6 g, 95%) as an liquid. ¹H NMR (300 MHz, CDCl₃): δ 6.98 (m, 1H), 6.41 (d, 2H), 5.27 (m, 1H), 5.10 (s, 2H), 3.43 (d, 2H), 1.80 (d, 6H), LCMS [M+H]⁺: 194; Purity (LCMS)>93%.

### Preparation of ethyl 3-oxo-3-(pyridin-4-yl)propanoate (Compound 35)

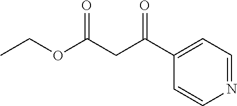

A mixture of ethyl isonicotinate (3.02 g), EtOAc (10 mL) and NaH (60%, 0.53 g) was heated to reflux for 3 h, cooled, diluted with water (20 mL) and was acidified with citric acid (5%). The aqueous phase was extracted with ethyl acetate, the organic extracts were washed with brine and dried over MgSO₄, filtered and concentrated in vacuum to afford Compound 35 (3.5 g, 85%) as an solid. ¹H NMR (300 MHz, DMSO): δ 8.40 (d, 2H), 7.58 (d, 2H), 5.03 (S, 2H), 3.93 (t, 2H), 1.14 (t, 3H), LCMS [M+H]⁺: 179; Purity (LCMS)>93%.

### Preparation of 7-hydroxy-8-(3-methylbut-2-enyl)-2-(pyridin-4-yl)-4H-chromen-4-one (Compound 36)

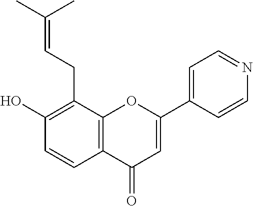

A mixture of Compound 34 (3.00 g), Compound 35 (3.00 g) and 1-phenoxybenzene (30 mL) was heated to reflux for 50 min, cooled, purified by flash chromatography to afford Compound 36 (300 mg, 6%) as an solid. ¹H NMR (300 MHz, DMSO): δ 10.81 (s, 1H), 8.82 (d, 2H), 8.00 (d, 2H), 7.80 (d, 2H), 7.13 (s, 1H), 7.00 (d, 1H), 5.25 (d, 1H), 3.61 (d, 2H), 1.640-1.79 (d, 6H), LCMS [M+H]⁺: 308; Purity (HPLC)>97%.

### Preparation of ethyl 3-oxo-3-(pyridin-3-yl)propanoate (Compound 37)

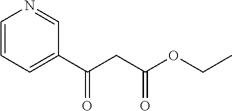

A mixture of ethyl nicotinate (2.26 g), EtOAc (20 mL) and NaOEt (11.4 g) was heated to reflux for 16 h. Water (20 mL) was added to the reaction mixture. The aqueous was acidified to pH 6 with HCl and extracted with ether. Solvent was removed in vacuo to give a brown oil which was purified by column chromatography (2:1, hexane-EtOAc) to give Compound 37 (1.01 g, 52%). ¹H NMR (300 MHz, DMSO): δ 8.90-9.10 (d, 1H), 8.20-8.80 (d, 1H), 8.00-8.25 (d, 1H), 7.30-7.40 (d, 1H), 4.26 (s, 2H), 1.30 (t, 3H), LCMS [M+H]⁺: 179; Purity (LCMS)>93%.

### Preparation of 7-hydroxy-8-(3-methylbut-2-enyl)-2-(pyridin-3-yl)-4H-chromen-4-one (Compound 38)

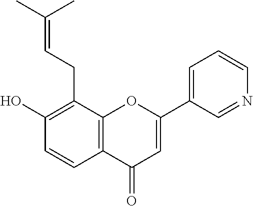

A mixture of Compound 34 (3.00 g), Compound 37 (3.00 g) and 1-phenoxybenzene (30 mL) was heated to reflux for 30 min. The reaction mixture was then cooled to room temperature and purified by flash chromatography to afford Compound 38 (200 mg, 4%) as a yellow solid. ¹H NMR (300 MHz, DMSO): δ 10.76 (s, 1H), 9.21 (s, 1H), 8.76 (d, 1H), 8.41 (d, 2H), 7.76 (d, 1H), 7.62 (d, 1H), 7.00 (t, 2H), 5.24 (d, 1H), 3.57 (d, 2H), 1.40-1.70 (d, 6H), LCMS [M+H]⁺: 308; Purity (HPLC)>97%.⁺.

Methods of Assessing Biological Activities

Activities of the compounds of the formula (I) of the present invention may be demonstrated by the following assays.

Expression of ER-α Variants in Human Breast Cancer Specimens

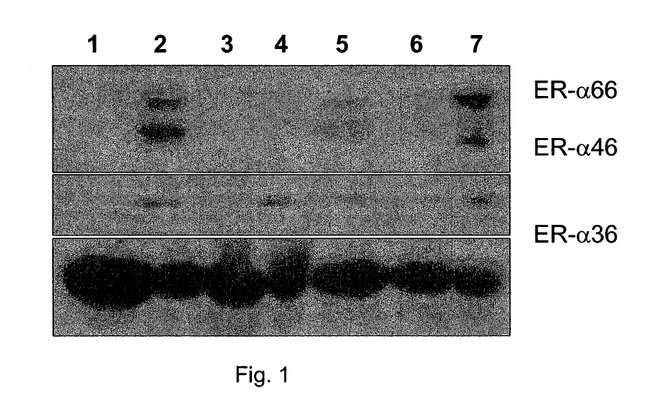

A membrane pre-blotted with human breast cancer tissues was purchased from ProSci Incorporated (Poway, Calif.). The membrane was probed with an anti-ER-α36 antibody that specifically recognizes ER-α36 and an HRP-conjugated secondary antibody, and visualized with enhanced chemiluminescence (ECL) detection reagents (Amersham Pharmacia Biotech). The same membrane was then stripped and detected with an anti-estrogen receptor-α antibody H222 (Novocastra Laboratories Ltd, UK) that recognizes all three subtypes of ER-α, ER-α66, ER-α46 and ER-α36. FIG. 1 shows that ER-α66, ER-α46 and ER-α36 are not expressed in normal breast tissue (Lane 1) but expressed in one specimen of infiltrating ductal carcinoma (Lane 2), one specimen of infiltrating lobular carcinoma (Lane 5), and non-invasive ductal carcinoma (Lane 7). In addition, ER-α36 is expressed in invasive ductal carcinoma (Lane 4) and another specimen of infiltrating lobular carcinoma (Lane 6). Lanes 2 and 3 had infiltrating ductal carcinoma from two different patients, respectively. Lanes 5 and 6 had infiltrating lobular carcinoma from two different patients, respectively. This result indicates that ER-α36 is not expressed in normal breast tissue but expressed in ER-negative breast cancer samples that do not express ER-α66 and ER-α46.

ER-α36 is Expressed in the ER-negative Breast Cancer Cell Line, MDA-MB-231

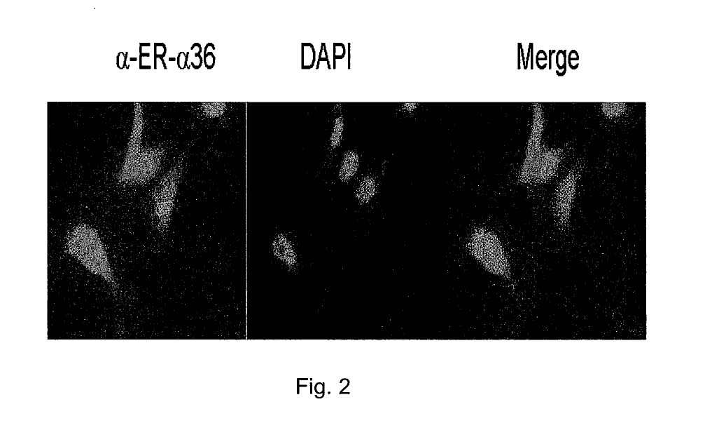

The MDA-MB-231 cell line is well-known for lacking ER-α66 and ER-α46 (Relevance of breast cancer cell lines as models for breast tumours: an update. Marc Lacroix, Guy Leclercq, Breast Cancer Research and Treatment 83: 249-289 (2004)). MDA-MB-231 cells were obtained from American Type Cell Culture (ATCC). MDA-MB-231 cells were grown on 8-well BIOCOAT chamber slides (BD Science Discovery Labware) in a 5% CO₂ atmosphere in Dulbecco's Modified Eagle's Medium (DMEM) and 10% fetal calf serum at 37° C. for 12 hours. Then the cells were washed twice with sterile Phosphate Buffered Saline (PBS) and fixed with 4% paraformaldehyde in PBS (pH 7.4) for 30 minutes at room temperature. After that, the cells were washed with PBS, permeabilized with 0.5% (v/v) Triton X-100 for 10 minutes. The cells were then washed with PBS again, and blocked with 3% serum in PBS at room temperature for 1 hour. The slides were incubated with an ER-α36 specific antibody or the same antibody preincubated with immunogen peptides that bind to the antibody for 30 minutes at room temperature for 1 hour and washed three times with PBS containing 0.5% Triton X-100 (PBST), then incubated with a fluorescein isothiocyanate (FITC)-conjugated secondary antibody. Finally, the slides were washed three times with PBST, one time with PBS, then coated with anti-fade medium (Molecular Probes, Eugene, Oreg.) and examined under a Nikon E600 Microscope and images were captured by the MRC-1024 confocal imaging system (Bio-Rad). FIG. 2 shows that MDA-MB-231 cells were stained positive by an anti-ER-α36 antibody. Incubation with the same antibody preincubated with the immunogen peptides did not show any staining, indicating the specificity of the antibody.

Cell Apoptosis Assay in ER-Negative Breast Cancer MDA-MB-231 Cells

MDA-MB-231 cells are maintained at 37° C. in a 5% CO₂ atmosphere in DMEM and 10% fetal calf serum. The cells are plated at a density of 1×10⁵ cells per 60-mm dish. MDA-MB-231 cells are treated with a test compound dissolved in DMSO at the concentrations of zero, 1 μM, 5 μM and 10 μM for a week. Treated cells are examined under a Nikon TS100 inverted microscope and photographed for morphological changes.

Cell Apoptosis Assay in ER-Positive Breast Cancer MCF7 Cells

MCF7 cell line is a breast cancer cell line that strongly expresses ER-α66, ER-α46 and ER-α36 (Relevance of breast cancer cell lines as models for breast tumours: an update. Marc Lacroix, Guy Leclercq, Breast Cancer Research and Treatment (2004) 83, 249-289; Wang et al., Proc. Natl. Acad. Sci. U.S.A. 103:9063-9068 (2006)). MCF7 cells obtained from ATCC are maintained in DMEM/F12 medium (Invitrogen) supplemented with 10% fetal calf serum at 37° C. in a 5% CO₂ atmosphere. The MCF7 cells are treated with the test compounds 6, 8, 14, 22, 27, 29, 30, 31, 36 and 38 at concentrations from 1 μM to 25 μM to test the effect of these compounds on MCF7 cell growth for 10 days. The control assay is conducted with MCF7 cells under the same assay conditions except that no test compound is added. Treated cells are examined under a Nikon TS100 inverted microscope and photographed for morphological changes. Table 1 shows the test results of the assays.

TABLE 1
The Cell Apoptosis Assay in ER-positive Breast Cancer MCF7 Cells

| | | Cell Number (×10⁴) | | | | | |
|---|---|---|---|---|---|---|---|
| | | | Tested Compound Concentration | | | | |
| | | | 1 | 5 | 10 | 15 | 25 |
| Compound | Cell lines | Control | μM | μM | μM | μM | μM |
| Compound 6 | MCF-7 | 24.9 | 18.35 | 8.97 | 3.95 | 3.9 | 3.87 |
| Compound 8 | MCF-7 | 24.9 | 28.75 | 15.35 | 10.1 | 6.45 | 0.25 |
| Compound 14 | MCF-7 | 27.5 | 30.05 | 14.54 | 2.51 | 0 | 0 |
| Compound 22 | MCF-7 | 23.4 | 39.0 | 21.6 | 15.6 | 4.2 | 0.6 |
| Compound 27 | MCF-7 | 23.4 | 59.4 | 45.2 | 5.4 | 1.05 | 0 |
| Compound 29 | MCF-7 | 23.4 | 27.9 | 30.1 | 24.3 | 23.4 | 12.3 |
| Compound 30 | MCF-7 | 22.4 | 21.1 | 10.8 | 5.8 | 3.6 | 1.5 |
| Compound 31 | MCF-7 | 22.4 | 24.0 | 25.5 | 27.25 | 9.6 | 7.5 |
| Compound 36 | MCF-7 | 26.6 | 25.6 | 14.8 | 10.65 | 3.65 | 1.4 |
| Compound 38 | MCF-7 | 26.6 | 27.8 | 19.66 | 13.8 | 10.1 | 2.6 |

Cell Apoptosis Assay in MCF7 Cells Over-Expressing ER-α36 and Tamoxifen Resistant MCF7 Cells

MCF7 cells over-expressing ER-α36 are made by stably transfecting MCF cells with an ER-α36 expression vector. The ER-α36 expression vector is constructed by cloning a 1.1-kb cDNA fragment of ER-α36 from pBluescript plasmid into a mammalian expression vector pCB6+ as described before (Wang et al., 2005, BBRC, 336:1023-1027). The constructed ER-α36 expression vector contains the cytomegalovirus (CMV) early promoter. MCF7 cells are transfected with the ER-α36 expression vector using the FuGene6 transfection reagent (Roche Molecular Biochemicals). Forty-eight hours after transfection, the cells are re-plated and selected with 500 μg/ml of G418 (Invitrogen) for about two weeks until colonies appear. The clones are then pooled and cultured to confirm the expression of ER-α36 by Western blot analysis.

Tamoxifen-resistant MCF7 cells are generated by incubating MCF7 cells in medium containing 5 μM Tamoxifen for three months. MCF7 cells that are still alive after three months are pooled and further cultured as Tamoxifen-resistant MCF7 cells. Western blot analysis is performed to identify cells highly expressing ER-α36.

The cell lines are maintained in DMEM/F12 medium supplemented with 10% fetal calf serum at 37° C. in a 5% CO₂ atmosphere. Cells are plated at a density of 1×10⁵ cells per 100-mm dish and treated with test compounds at concentrations of zero to 10 μM for two weeks. The numbers of survived cells after two weeks are counted. Five dishes of cells are counted for each concentration point.

In Vivo Assay:

In Vivo Efficacy in Inhibiting Growth of ER-Positive and ER-Negative Breast Cancer Xenografts in Nude Mice

Test compound for administration to animals is prepared in corn oil (20 mg/mL). The drug solution is stored at 4° C. and ready to be used for animal administration. The drug solution is administered to mice using the gavage technique.

Tumor formation is assayed in female athymic nude mice (6 weeks old). MCF7 cells or MDA-MB-231 cells at the concentration of 1×10⁷ cells in 200 μl Matrigel (BD Biosciences) are injected into the mice by the mammary fatpad injection. A group of 5 mice are injected with each type of breast cancer cells. For MCF7 cells, inoculation is performed 5 days after subcutaneous implantation of 1.7 mg/60-day release E2β pellets (a slow release E2β pellet that can release a certain amount of E2β every day for 60 days). Animals with tumor size about 0.5 cm in diameter are administered with test compounds in corn oil using the gavage technique with an animal feeding needle. For the mice inoculated with MCF7 cells, each is feed with 5 mg of test compound every other day for 15 days. For the mice inoculated with MDA-MB-231 cells, each is feed with 5 mg of test compound every other day for 30 days. Tumor disappearance is determined by palpation, and the sizes of tumors are also monitored by measuring two perpendicular diameters with vernier calipers every other day and photographed.

The In Vivo Efficacy in Inhibiting Growth of Human Breast Cancer BCAP-37 Cells Xenografts in Nude Mice Model

Nude mice with breast cancer xenografts are treated with the test compounds 5, 6, 8, 14, 22, 36 and 38 to test their effect on inhibiting tumor growth. Tumor tissues are taken from nude mice bearing BCAP-37 breast cancer and cut into small pieces. Several pieces of the tumor tissues are implanted into the armpit under the right front limb of female nude mice. After the implantation, the mice are fed with E2β solution once every day at the dosage of 7 μg per mouse for 6 days to stimulate tumor growth in the receiving mice. Starting on the seventh day, the mice are fed with the test compounds at the dosage of 35 mg/kg. Tamoxifen is used as a positive control. Olive oil is used as a negative control. The test compounds are prepared as an olive oil solution (20 mg/mL). The mice are given the test compounds and Tamoxifen at the dosage of 35 mg/kg or olive oil once every day for 15 days. Then the mice are sacrificed and the tumor tissues are dissected from the mice and weighed. The tumor growth inhibition rate is a percentage calculated using the formula: tumor growth inhibition rate=(average weight of the tumor in the negative control−average weight of the tumor treated with test compound)/average weight of the tumor in the negative control. The experiment results are shown in Table 2 below.

TABLE 2
The In Vivo Efficacy of Test Compounds in Inhibiting
Growth of Human Breast Cancer BCAP-37 Cells

| Compounds | Inhibition rate of tumor growth (%) |
|---|---|
| Negative Control | 0 |
| Tamoxifen | 40-50 |
| Compound 5 | 26 |
| Compound 6 | 44 |
| Compound 8 | 62 |
| Compound 14 | 37 |
| Compound 22 | 66 |
| Compound 36 | 56 |
| Compound 38 | 24 |

The In Vivo Efficacy in Inhibiting Growth of Murine Cervical Cancer U14 Cells Xenografts in ICR Mice

Five ml of abdominal fluid is drawn from the lower abdomen of ICR mice containing cervical cancer U14 cell line. The fluid is diluted at the ratio of 1:5 with saline. 0.2 ml of the diluted abdominal fluid is injected subcutaneously at the right front limb and chest of female ICR mice. After the implantation, the mice are treated right away with test compound, Tamoxifen and olive oil, respectively. The mice are given the test compound at different dosages, Tamoxifen or olive oil once every day for a period of 14 days. Then the mice are sacrificed and the tumor tissues are dissected from the mice and weighed. Ten female ICR mice are used for each test compounds and dosages. The tumor growth inhibition rates of the mice treated with the test compound, Tamoxifen and olive oil are calculated and compared.

We claim:

1. A method of preventing or treating a disease involving abnormal cell proliferation in a subject comprising administering to the subject a therapeutically effective amount of a pharmaceutical composition comprising an isolated compound having the structure of formula (I) as shown below, 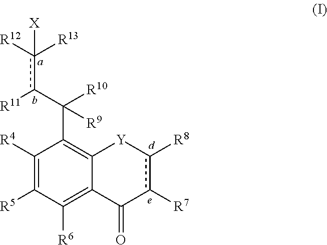
wherein:
X═H, OR¹ or NR²R³; Y═NR, O; R¹, R², R and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R⁸ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, aryl (including substituted aryl), (C₆-C₁₀)aryloxy, heteroaryl (including substituted heteroaryl), (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹², and R¹³ are hydrogen, halo, hydroxyl, (C₁-C₆)alkyl;
when R, R¹, R², R³, R⁴, R⁵, R⁶, R⁷, R⁸, R⁹, R¹⁰, R¹¹, R¹², R¹³ are (C₁-C₆)alkyl group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₉)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

2. The method of claim 1 wherein the compound has the structure of formula IA1 as shown below, 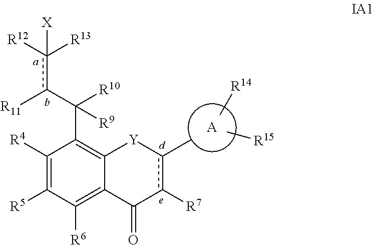
Wherein: 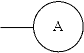
is (C₆-C₁₀)aryl, (C₅-C₈)heteroaryl, X═H, OR¹ or NR²R³; Y═NR, O; wherein R, R¹, R² and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R¹⁴ and R¹⁵ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl (including substituted aryl), (C₆-C₁₀)aryloxy, (C₅-C₈)heteroaryl (including substituted heteroaryl), (C₅-C₉)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹² and R¹³ are hydrogen, halo, hydroxyl, (C₁-C₆)alkyl;
When R, R¹, R², R³, R⁴, R⁵, R⁶, R⁷, R⁹, R¹⁰, R¹¹, R¹², R¹³, R¹⁴ and R¹⁵ are (C₁-C₆) group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₈)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

3. The method of claim 2 wherein the compound has the structure of formula IA2 as shown below, 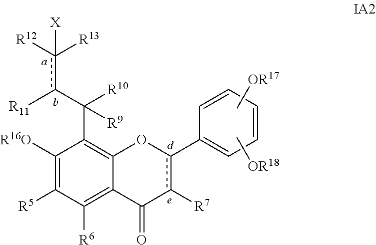
Wherein R¹⁶, R¹⁷ and R¹⁸ and are independently hydrogen, (C₁-C₆)alkyl, (C₂-C₆)alkenyl, (C₂-C₆)alkynyl; the bond between carbon a and b or d and e may be single or double bond. R⁵, R⁶, R⁷, R⁹, R¹⁰, R¹¹, R¹², R¹³ and X are as defined above.

4. The method of claim 3 wherein the compound has the structure of formula IA3 as shown below, 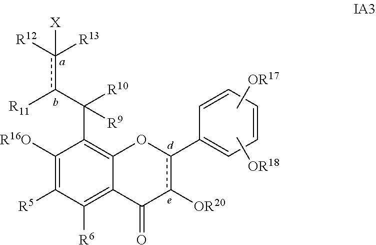
Wherein R¹⁶, R⁷, R¹⁸ and R²⁰ are independently hydrogen, (C₁-C₆)alkyl; R⁹, R¹⁰, R¹¹, R¹², R¹³ are independently hydrogen, (C₁-C₆)alkyl; and X are H, OR¹ or NR²R³, R¹, R² and R³ are independently hydrogen, (C₁-C₆)alkyl or R²R³ together as —(CH₂)ₙ—, n=2 to 8; the bond between carbon a and b or d and e may be single or double bond. R⁵ and R⁶ are defined as above.

5. The method of claim 4 wherein the compound has the structure of formula IA4 as shown below, 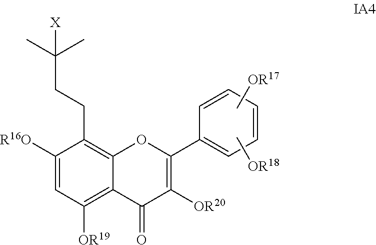
Wherein R¹⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R²R³ together as —(CH₂)ₙ—, n=2 to 5.

6. The method of claim 5 wherein the compound has the structure of formula IA5 as shown below, 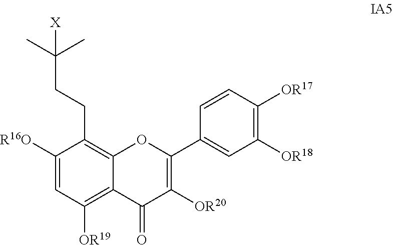
Wherein R¹⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R₂R₃ together as —(CH₂)ₙ—, n=2 to 5.

7. The method of claim 6 wherein the compound has the structure of formula IA6 as shown below, 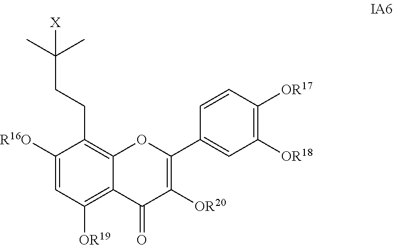
Wherein R¹⁶, R¹⁷, R¹⁸, R¹⁹ and R²⁰ are independently hydrogen, (C₁-C₆)alkyl; and X are OH or NH₂.

8. The method of claim 1 wherein the compound is selected from the group consisting of 8-(3-amino-3-methylbutyl)-3,5,7-trihydroxy-2-(4-hydroxyphenyl)-4H-chromen-4-one, 8-(3-amino-3-methylbutyl)-3,5,7-trihydroxy-2-(4-methoxyphenyl)-4H-chromen-4-one, 8-(3-amino-3-methylbutyl)-5,7-dihydroxy-3-methoxy-2-(4-methoxyphenyl)-4H-chromen-4-one, 5,7-dihydroxy-8-(3-hydroxy-3-methylbutyl)-3-methoxy-2-(4-methoxyphenyl)-4H-chromen-4-one, 5,7-dihydroxy-8-(3-hydroxy-3-methylbutyl)-3-methoxy-2-(3,4-dimethoxyphenyl)-4H-chromen-4-one, 5-hydroxy-8-(3-hydroxy-3-methylbutyl)-3,7-dimethoxy-2-(3,4-dimethoxyphenyl)-4H-chromen-4-one, 8-(3-amino-3-methylbutyl)-5-hydroxy-3,7-dimethoxy-2-(3,4-dimethoxyphenyl)-4H-chromen-4-one, 3-(2-(piperidin-1-yl)ethoxy)-5,7-dihydroxy-8-(3-hydroxy-3-methylbutyl)-2-(4-methoxyphenyl)-4H-chromen-4-one, 3-(2-(piperidin-1-yl)ethoxy)-8-(3-amino-3-methylbutyl)-5,7-dihydroxy-2-(4-methoxyphenyl)-4H-chromen-4-one, 2-(4-chlorophenyl)-3,5,7-trihydroxy-8-(3-hydroxy-3-methylbutyl)-4H-chromen-4-one, 2-(4-chloro-3-methoxyphenyl)-3,5,7-trihydroxy-8-(3-hydroxy-3-methylbutyl)-4H-chromen-4-one, 8-(3-amino-3-methylbutyl)-3,5,7-trihydroxy-2-(pyridin-3-yl)-4H-chromen-4-one, 8-(3-amino-3-methylbutyl)-3,5,7-trihydroxy-2-(pyridin-2-yl)-4H-chromen-4-one, 8-(3-amino-3-methylbutyl)-5,7-dihydroxy-3-methoxy-2-(5-methoxypyridin-2-yl)-4H-chromen-4-one, 5,7-dihydroxy-8-(3-hydroxy-3-methylbutyl)-3-methoxy-2-(6-methoxypyridin-3-yl)-4H-chromen-4-one, 5,7-dihydroxy-3-methoxy-8-(3-methylbut-2-enyl)-2-(pyridin-3-yl)-4H-chromen-4-one, 2-(6-(dimethylamino)pyridin-3-yl)-5,7-dihydroxy-3-methoxy-8-(3-methylbut-2-enyl)-4H-chromen-4-one, 2,3-dihydro-5,7-dihydroxy-2-(4-methoxyphenyl)-8-(3-methylbut-2-enyl)quinolin-4(1H)-one, 2,3-dihydro-7-hydroxy-2-(4-methoxyphenyl)-8-(3-methylbut-2-enyl)quinolin-4(1H)-one, 7-hydroxy-2-(4-methoxyphenyl)-8-(3-methylbut-2-enyl)quinolin-4(1H)-one, 5,7-dihydroxy-2-(4-methoxyphenyl)-8-(3-methylbut-2-enyl)quinolin-4(1H)-one, 8-(3-amino-3-methylbutyl)-5,7-dihydroxy-2-(4-hydroxyphenyl)-4H-chromen-4-one, 8-(3-amino-3-methylbutyl)-5,7-dihydroxy-2-(4-methoxyphenyl)-4H-chromen-4-one, 8-(3-amino-3-methylbutyl)-2,3-dihydro-5,7-dihydroxy-2-(4-methoxyphenyl)chromen-4-one, 5,7-dihydroxy-8-(3-hydroxy-3-methylbutyl)-2-(4-methoxyphenyl)-4H-chromen-4-one, 5,7-dihydroxy-8-(3-hydroxy-3-methylbutyl)-2-(3,4-dimethoxyphenyl)-4H-chromen-4-one, 5-hydroxy-8-(3-hydroxy-3-methylbutyl)-7-methoxy-2-(3,4-dimethoxyphenyl)-4H-chromen-4-one, 8-(3-amino-3-methylbutyl)-5-hydroxy-7-methoxy-2-(3,4-dimethoxyphenyl)-4H-chromen-4-one, 2,3-dihydro-5,7-dihydroxy-8-(3-hydroxy-3-methylbutyl)-2-(4-methoxyphenyl)chromen-4-one, 2-(4-aminophenyl)-5,7-dihydroxy-8-(3-hydroxy-3-methylbutyl)-4H-chromen-4-one, 2-(4-chlorophenyl)-5,7-dihydroxy-8-(3-hydroxy-3-methylbutyl)-4H-chromen-4-one, 2-(4-chloro-3-methoxyphenyl)-5,7-dihydroxy-8-(3-hydroxy-3-methylbutyl)-4H-chromen-4-one, 2-(4-aminophenyl)-2,3-dihydro-5,7-dihydroxy-8-(3-hydroxy-3-methylbutyl)chromen-4-one, 2-(4-chlorophenyl)-2,3-dihydro-5,7-dihydroxy-8-(3-hydroxy-3-methylbutyl)chromen-4-one, 2-(4-chloro-3-methoxyphenyl)-2,3-dihydro-5,7-dihydroxy-8-(3-hydroxy-3-methylbutyl)chromen-4-one, 7-hydroxy-8-(3-methylbut-2-enyl)-2-(pyridin-4-yl)-4H-chromen-4-one, 7-hydroxy-8-(3-methylbut-2-enyl)-2-(pyridin-3-yl)-4H-chromen-4-one.

9. The method of claim 1 wherein the subject is mammal.

10. The method of claim 9 wherein the mammal is human.

11. The method of claim 10 wherein the administering is oral, buccal, sublingual, ocular, topical, parenteral, rectal, intracistemal, intravaginal, intraperitoneal, intravesical, or nasal.

12. The method of claim 11 wherein the administering is parenteral.

13. The method of claim 1 wherein the disease is selected from the group consisting of breast cancer, colon cancer, leukemia, liver cancer, lung cancer, myeloma, ovary cancer, prostate cancer, stomach cancer, or uterus cancer.

14. The method of claim 13 wherein the disease is breast cancer.

15. A method of preventing or treating a disease mediated by ER-α in a subject comprising administering to the subject a therapeutically effective amount of a pharmaceutical composition comprising a compound having the structure of Formula I as shown below, 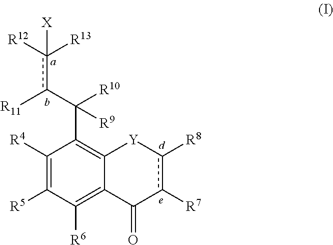
wherein:
X═H, OR¹ or NR²R³; Y═NR, O; R¹, R², R and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R⁸ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, aryl (including substituted aryl), (C₆-C₁₀)aryloxy, heteroaryl (including substituted heteroaryl), (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹², and R¹³ are hydrogen, halo, hydroxyl, (C₁-C₆)alkyl;
when R, R¹, R², R³, R⁴, R⁵, R⁶, R⁷, R⁸, R⁹, R¹⁰, R¹¹, R¹², R¹³ are (C₁-C₆)alkyl group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₈)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

16. The method of claim 15 wherein the compound has the structure of formula IA1 as shown below, 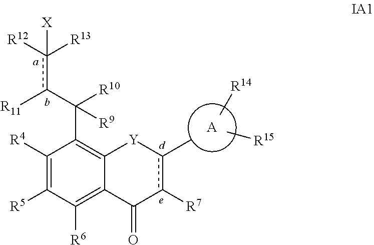
Wherein: 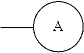
is (C₆-C₁₀)aryl, (C₅-C₉)heteroaryl, X═H, OR¹ or NR²R³; Y═NR, O; wherein R, R¹, R² and R³ are independently hydrogen, (C₁-C₆)alkyl or R²R³ together as —(CH₂)ₙ—, n=2 to 8; the bond between carbon a and b or d and e may be single or double bond. R⁴, R⁵, R⁶, R⁷, R¹⁴ and R¹⁵ are independently hydrogen, halo, hydroxyl, amino, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl (including substituted aryl), (C₆-C₁₀)aryloxy, (C₅-C₈)heteroaryl (including substituted heteroaryl), (C₅-C₉)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl. R⁹, R¹⁰, R¹¹, R¹² and R¹³ are hydrogen, halo, hydroxyl, (C₁-C₆)alkyl;
When R, R¹, R², R³, R⁴, R⁵, R⁶, R⁷, R⁹, R¹⁰, R¹¹, R¹², R¹³, R¹⁴ and R¹⁵ are (C₁-C₆)alkyl group, each carbon atom of the (C₁-C₆)alkyl group may be optionally substituted with one to three substituents independently selected from hydroxyl, halo, (C₁-C₆)alkyl, (C₂-C₆)alkenyl, (C₂-C₆)alkynyl, (C₁-C₆)alkoxy, (C₁-C₆)alkyl-(C═O)—, formyl, formamidyl, cyano, nitro, HO—(C═O)—, (C₁-C₆)alkoxycarbonyl, aminocarbonyl, amino(C₁-C₆)alkyl, N-(C₁-C₆)alkylaminocarbonyl, N,N—[(C₁-C₆)alkyl]₂aminocarbonyl, N-(C₆-C₁₀)arylaminocarbonyl, N,N—[(C₆-C₁₀)aryl]₂aminocarbonyl, N-(C₁-C₆)alkyl-N-(C₁-C₆)alkylaminocarbonyl, N-(C₁-C₆)alkyl-N-(C₆-C₁₀)arylaminocarbonyl, (C₆-C₁₀)aryl, (C₆-C₁₀)aryloxy, (C₅-C₈)heteroaryl, (C₅-C₈)heteroaryloxy, morpholino-carbonyl, (C₁-C₆)alkoxyaminocarbonyl, (C₁-C₆)alkyl-carbonylamino, (C₃-C₈)cycloalkyl, (C₃-C₈)cycloalkyl-methyl, (C₃-C₈)heterocycloalkyl, (C₃-C₈)heterocycloalkyl-methyl.

17. The method of claim 16 wherein the compound has the structure of formula IA2 as shown below, 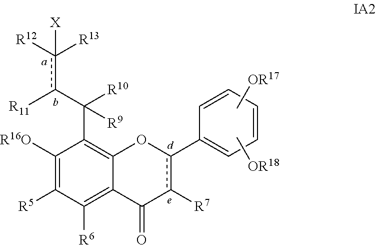
Wherein R¹⁶, R¹⁷ and R¹⁸ and are independently hydrogen, (C₁-C₆)alkyl, (C₂-C₆)alkenyl, (C₂-C₆)alkynyl; the bond between carbon a and b or d and e may be single or double bond. R⁵, R⁶, R⁷, R⁹, R¹⁰, R¹¹, R¹², R¹³ and X are as defined above.

18. The method of claim 17 wherein the compound has the structure of formula IA3 as shown below, 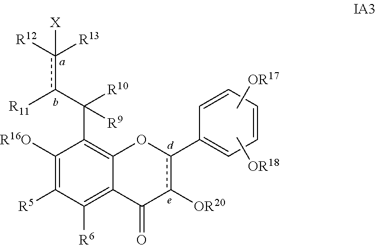
Wherein R¹⁶, R¹⁷, R¹⁸ and R²⁰ are independently hydrogen, (C₁-C₆)alkyl; R⁹, R¹⁰, R¹¹, R¹², R¹³ are independently hydrogen, (C₁-C₆)alkyl; and X are H, OR¹ or NR²R³, R¹, R² and R³ are independently hydrogen, (C₁-C₆)alkyl or R²R³ together as —(CH₂)ₙ—, n=2 to 8; the bond between carbon a and b or d and e may be single or double bond. R⁵ and R⁶ are defined as above.

19. The method of claim 18 wherein the compound has the structure of formula IA4 as shown below, 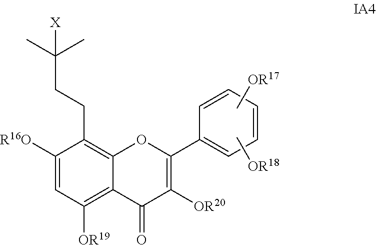
Wherein R¹⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R²R³ together as —(CH₂)ₙ—, n=2 to 5.

20. The method of claim 19 wherein the compound has the structure of formula IA5 as shown below, 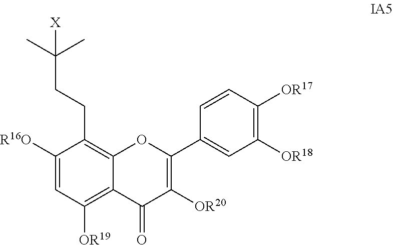
Wherein R¹⁶, R¹⁷, R¹⁸, R¹⁹ and R²⁰ are independently hydrogen, (C₁-C₆)alkyl; and X are OR¹ or NR²R³, R¹, R² and R³ are independently hydrogen, (C₁-C₆)alkyl or R₂R₃ together as —(CH₂)ₙ—, n=2 to 5.

21. The method of claim 20 wherein the compound has the structure of formula IA6 as shown below, 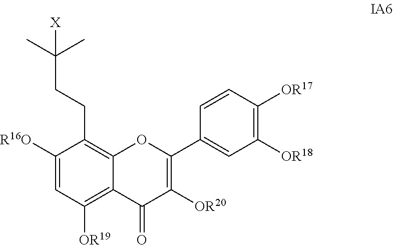
Wherein R¹⁶, R¹⁷, R¹⁸, R¹⁹ and R²⁰ are independently hydrogen, (C₁-C₆)alkyl; and X are OH or NH₂.

22. The method of claim 15 wherein the subject is mammal.

23. The method of claim 22 wherein the mammal is human.

24. The method of claim 15 wherein the administering is oral, buccal, sublingual, ocular, topical, parenteral, rectal, intracisternal, intravaginal, intraperitoneal, intravesical, or nasal.

25. The method of claim 24 wherein the administering is parenteral.

26. The method of claim 25 wherein the parenteral administration is intravenous, intramuscular, or subcutaneous, intravascular or infusion.

27. The method of claim 15 wherein the disease is osteoporosis.

28. The method of claim 15 wherein the compound is selected from the group consisting of 8-(3-amino-3-methylbutyl)-3,5,7-trihydroxy-2-(4-hydroxyphenyl)-4H-chromen-4-one, 8-(3-amino-3-methylbutyl)-3,5,7-trihydroxy-2-(4-methoxyphenyl)-4H-chromen-4-one, 8-(3-amino-3-methylbutyl)-5,7-dihydroxy-3-methoxy-2-(4-methoxyphenyl)-4H-chromen-4-one, 5,7-dihydroxy-8-(3-hydroxy-3-methylbutyl)-3-methoxy-2-(4-methoxyphenyl)-4H-chromen-4-one, 5,7-dihydroxy-8-(3-hydroxy-3-methylbutyl)-3-methoxy-2-(3,4-dimethoxyphenyl)-4H-chromen-4-one, 5-hydroxy-8-(3-hydroxy-3-methylbutyl)-3,7-dimethoxy-2-(3,4-dimethoxyphenyl)-4H-chromen-4-one, 8-(3-amino-3-methylbutyl)-5-hydroxy-3,7-dimethoxy-2-(3,4-dimethoxyphenyl)-4H-chromen-4-one, 3-(2-(piperidin-1-yl)ethoxy)-5,7-dihydroxy-8-(3-hydroxy-3-methylbutyl)-2-(4-methoxyphenyl)-4H-chromen-4-one, 3-(2-(piperidin-1-yl)ethoxy)-8-(3-amino-3-methylbutyl)-5,7-dihydroxy-2-(4-methoxyphenyl)-4H-chromen-4-one, 2-(4-chlorophenyl)-3,5,7-trihydroxy-8-(3-hydroxy-3-methylbutyl)-4H-chromen-4-one, 2-(4-chloro-3-methoxyphenyl)-3,5,7-trihydroxy-8-(3-hydroxy-3-methylbutyl)-4H-chromen-4-one, 8-(3-amino-3-methylbutyl)-3,5,7-trihydroxy-2-(pyridin-3-yl)-4H-chromen-4-one, 8-(3-amino-3-methylbutyl)-3,5,7-trihydroxy-2-(pyridin-2-yl)-4H-chromen-4-one, 8-(3-amino-3-methylbutyl)-5,7-dihydroxy-3-methoxy-2-(5-methoxypyridin-2-yl)-4H-chromen-4-one, 5,7-dihydroxy-8-(3-hydroxy-3-methylbutyl)-3-methoxy-2-(6-methoxypyridin-3-yl)-4H-chromen-4-one, 5,7-dihydroxy-3-methoxy-8-(3-methylbut-2-enyl)-2-(pyridin-3-yl)-4H-chromen-4-one, 2-(6-(dimethylamino)pyridin-3-yl)-5,7-dihydroxy-3-methoxy-8-(3-methylbut-2-enyl)-4H-chromen-4-one, 2,3-dihydro-5,7-dihydroxy-2-(4-methoxyphenyl)-8-(3-methylbut-2-enyl)quinolin-4(1H)-one, 2,3-dihydro-7-hydroxy-2-(4-methoxyphenyl)-8-(3-methylbut-2-enyl)quinolin-4(1H)-one, 7-hydroxy-2-(4-methoxyphenyl)-8-(3-methylbut-2-enyl)quinolin-4(1H)-one, 5,7-dihydroxy-2-(4-methoxyphenyl)-8-(3-methylbut-2-enyl)quinolin-4(1H)-one, 8-(3-amino-3-methylbutyl)-5,7-dihydroxy-2-(4-hydroxyphenyl)-4H-chromen-4-one, 8-(3-amino-3-methylbutyl)-5,7-dihydroxy-2-(4-methoxyphenyl)-4H-chromen-4-one, 8-(3-amino-3-methylbutyl)-2,3-dihydro-5,7-dihydroxy-2-(4-methoxyphenyl)chromen-4-one, 5,7-dihydroxy-8-(3-hydroxy-3-methylbutyl)-2-(4-methoxyphenyl)-4H-chromen-4-one, 5,7-dihydroxy-8-(3-hydroxy-3-methylbutyl)-2-(3,4-dimethoxyphenyl)-4H-chromen-4-one, 5-hydroxy-8-(3-hydroxy-3-methylbutyl)-7-methoxy-2-(3,4-dimethoxyphenyl)-4H-chromen-4-one, 8-(3-amino-3-methylbutyl)-5-hydroxy-7-methoxy-2-(3,4-dimethoxyphenyl)-4H-chromen-4-one, 2,3-dihydro-5,7-dihydroxy-8-(3-hydroxy-3-methylbutyl)-2-(4-methoxyphenyl)chromen-4-one, 2-(4-aminophenyl)-5,7-dihydroxy-8-(3-hydroxy-3-methylbutyl)-4H-chromen-4-one, 2-(4-chlorophenyl)-5,7-dihydroxy-8-(3-hydroxy-3-methylbutyl)-4H-chromen-4-one, 2-(4-chloro-3-methoxyphenyl)-5,7-dihydroxy-8-(3-hydroxy-3-methylbutyl)-4H-chromen-4-one, 2-(4-aminophenyl)-2,3-dihydro-5,7-dihydroxy-8-(3-hydroxy-3-methylbutyl)chromen-4-one, 2-(4-chlorophenyl)-2,3-dihydro-5,7-dihydroxy-8-(3-hydroxy-3-methylbutyl)chromen-4-one, 2-(4-chloro-3-methoxyphenyl)-2,3-dihydro-5,7-dihydroxy-8-(3-hydroxy-3-methylbutyl)chromen-4-one, 7-hydroxy-8-(3-methylbut-2-enyl)-2-(pyridin-4-yl)-4H-chromen-4-one, 7-hydroxy-8-(3-methylbut-2-enyl)-2-(pyridin-3-yl)-4H-chromen-4-one
